(19) United States
(12) Patent Application Publication
(10) Pub. No.: US 2001/0031253 A1
(43) Pub. Date: Oct. 18, 2001

(54) Autologous immune cell therapy: cell compositions, methods and applications to treatment of human disease

(76) Inventor: Micheal L. Gruenberg, Poway, CA (US)

Correspondence Address: Stephanie Seidman, Heller Ehrman White & McAuliffe LLP, 4250 Executive Square, 7th Floor, La Jolla, CA, 92037, US

(21) Appl. No.: 09/824,906

(22) Filed: Apr. 2, 2001

Related U.S. Application Data
(62) Division of application No. 08/700,565, filed on Jul. 25, 1996.
(62) Division of application No. PCT/US96/12170, filed on Jul. 24, 1996.
(60) Provisional application No. 60/044,693, filed on Jul. 26, 1995.

(51) Int. Cl. C12N 5/08

(52) U.S. Cl. 424/93.1; 435/372.3

(57) ABSTRACT

Compositions containing clinically relevant numbers of immune cells that have been isolated from a patient differentiated and/or expanded ex vivo. Methods for treating or preventing disease or otherwise altering the immune status of the patient by reinfusing such cells into the donor are also provided. Methods for expanding and/or immune cells, including effector cells, in the absence of exogenous IL-2, and for administering the cells in the absence of co-infused IL-2 are also provided.

### RELATED APPLICATIONS

This application is a divisional of U.S. application Ser. No. 08/700,565 to Micheal Gruenberg, entitled AUTOLOGOUS IMMUNE CELL THERAPY: CELL COMPOSITIONS, METHODS AND APPLICATIONS TO TREATMENT OF HUMAN DISEASE, filed Jul. 25, 1996, which application claims the benefit of priority under 35 U.S.C. §119(e) to provisional application No. 60/044,693, filed on Jul. 26, 1995 to Micheal Gruenberg, entitled PROCESS FOR PRODUCING EFFECTOR IMMUNE CELLS FOR USE IN ADOPTIVE CELLULAR IMMUNOTHERAPY, which provisional application was filed as U.S. application Ser. No. 08/506,668 on Jul. 26, 1995, and converted to a provisional application.

This application is also a continuation-in-part of International PCT application No. PCT/US96/12170, filed Jul. 24, 1996, by CellTherapy, Inc. and Micheal Gruenberg, entitled AUTOLOGOUS IMMUNE CELL THERAPY: CELL COMPOSITIONS, METHODS AND APPLICATIONS TO TREATMENT OF HUMAN DISEASE.

This application is also related to U.S. application Ser. No. 08/759,645, filed Dec. 5, 1996, now U.S. Pat. No. 5,763,261, to Micheal Gruenberg, entitled CELL GROWING DEVICE FOR IN VITRO CELL POPULATION EXPANSION, which is a continuation of U.S. application Ser. No. 08/506,173, filed Jul. 26, 1995, now U.S. Pat. No. 5,637,070, to Micheal Gruenberg, entitled CELL GROWING DEVICE FOR IN VITRO CELL POPULATION EXPANSION. The subject matter of each of U.S. application Ser. Nos. 08/700,565, 08/506,668, 08/506,173, 08/759,645 and International PCT application No. PCT/US96/12170 is herein incorporated by reference in its entirety.

### FIELD OF INVENTION

This invention is directed to methods of adoptive immunotherapy. In particular, methods of autologous cell therapy are provided. Compositions containing substantially homogeneous populations of functionally or phenotypically defined immune cells that have been isolated from a patient, differentiated and/or expanded ex vivo are provided. Uses of such compositions for treating or preventing disease or otherwise altering the immune status of the patient by reinfusing such cells are also provided.

### BACKGROUND OF INVENTION

T lymphocytes are immune cells that are primarily responsible for protection against intracellular pathogens and suppression or elimination of certain tumors. Mature T lymphocytes, which all express the CD3 cell surface antigen, are subdivided into two subtypes, based on expression of either the CD4 or CD8 surface antigen. CD4⁺ T cells recognize antigen presented in association with class II major histocompatibility complex (MHC) molecules. CD4⁺ cells are generally involved in regulatory functions in immune responses by virtue of the cytokines they produce. These cytokines, such as IL-2, mediate an immune cell attack on a pathogen or an antibody attack against an invading organism.

CD8⁺ T cells recognize antigen presented in association with class I MHC molecules. CD8⁺ cells are involved in effector functions in immune responses, such as cytotoxic destruction of cells bearing foreign antigens. The cells that mediate these responses are designated cytotoxic T lymphocytes (CTLs). These cells, which are generally CD8⁺ cells (although some are CD4⁺) represent a mechanism for resistance to viral infections and tumors. The effector function of CTLs is dependent upon the cytokine production from CD4⁺ regulatory cells.

Adoptive Immunotherapy

Adoptive immunotherapy is an experimental treatment method designed to boost a patient's immune response against a virus or a tumor. The method involves the removal of immune cells from an individual, the forming of effector cells outside the body (ex vivo), the expansion of the cells to clinically-relevant numbers and the re-infusion of the cells into the patient. Adoptive immunotherapy protocols have not been made commercially available and are not in widespread use because of the extreme toxicities associated with the infusion of the interleukin-2 (IL-2) with the cells. IL-2 is used in these protocols to cause the differentiation and/or expansion of effector immune cells. Immune cells cultivated in IL-2, however, become dependent on the cytokine for continued viability and effector function, thus necessitating the infusion of IL-2 together with the effector cells. All adoptive immunotherapy protocols involving differentiated effector cells incorporate the use of IL-2.

The severe toxicity associated with the use of IL-2 has limited the application of adoptive immunotherapy to the treatment of terminally-ill cancer patients and the treatment of viral infections in AIDS patients.

Adoptive Immunotherapy and the Use Thereof for Treating Cancer

The first attempts at adoptive immunotherapy in humans employed lymphokine activated killer (LAK) cells, which are immune effector cells functionally defined by their ability to lyse fresh tumors. LAK cells are produced when peripheral blood mononuclear cells are exposed to high concentrations of IL-2 ex vivo [see, e.g., Grimm, et a. (1982) J. Exp. Med. 155:1832]. To produce LAK cells for use in treating cancer patients [see, U.S. Pat. No. 4,690,915], leukocytes are removed from a cancer patient and exposed to high levels of IL-2 for 3-6 days, which causes a portion of the cells to differentiate into LAK cells. The resulting heterogeneous population of cells is reinfused to the donor concomitant with a high systemic dose of IL-2. As noted, the high systemic doses of IL-2 are highly toxic and not well tolerated.

Methods in which the potency of LAK cells is increased have been developed. It has been observed [see, e.g., U.S. Pat. No. 4,849,329] that the addition of an L-amino acid with IL-2 during the ex vivo differentiation step increases the LAK activity of the resulting cells 4-5 fold. Administration of LAK cells with IL-2 and an ornithine decarboxylase inhibitor enhances the effectiveness of the treatment [see, U.S. Pat. No. 5,002,879]. Exposure of lymphocytes to an anti-CD3 monoclonal antibody (mAb) during the LAK differentiation stage of the process produces effector cells with enhanced anti-tumor activity [U.S. Pat. No. 5,326,763], and use of IL-7, with or without IL-2, in the LAK differentiation step can also produce more potent LAK effector cells [see, U.S. Pat. No. 5,229,115]. The administration of GM-CSF with IL-2 has also been reported to cause an increase in LAK activity [see Takahashi, et al. (1995) Jap. J. Cancer Res. 86:861]. All protocols, however, require administration of IL-2.

Early clinical results of adoptive immunotherapy using LAK cells in terminally-ill cancer patients, particularly those with malignant melanoma, had reported response rates of 21-44% [see, e.g., Rosenberg et al. (1985) N. Engl. J. Med. 313:1485 and Rosenberg et al. (1987) N. Engl. J. Med. 316:889]. Results of more recent phase 11 clinical studies, while still showing promise, have produced a broad range of response rates from 0-33% [see, e.g., Dillman, et al. (1991) J. Clin. Oncol. 9:1233. Thompson, J. A. et al. (1992) J. Clin. Oncol. 10:960); Foon, et al. (1992) J. Immunother. 11:1984 and Koretz, et al. (1991) Arch. Surg. 126:898]. The differences in response rates are attributed, partly, to variations in dosages of LAK cells and IL-2 administrated, and the differences in tumor-killing activities of the heterogeneous populations of LAK cells generated from different patients.

Methods for generating a relatively homogenous population of LAK cells for adoptive immunotherapy have been developed [see, U.S. Pat. No. 5,057,423]. The process described in U.S. Pat. No. 5,057,423 involves first purifying a population of LAK progenitor cells (LGL) from the peripheral blood mononuclear cells. These LGL are then exposed to IL-2, which causes a majority of the LGL to differentiate into LAK cells. The resulting effector cells, known as A-LAK, have been shown to be effective in killing human carcinoma in nude mice [see, Sacchi (1991) et al. Int. J. Cancer 47:784; Boiardi, et al. (1994) Cancer Immunol. Immunoth. 39:193]. It is exceedingly difficult, however, to produce sufficient numbers of A-LAK from humans. Even with the use of feeder cells to improve ex vivo expansion, A-LAK cultures from approximately 60% of cancer patients demonstrated inadequate expansion [see, Sedlmayr, et al. (1991) J. Immunother. 10:336].

Another adoptive immunotherapy protocol involves the administration of autologous tumor infiltrating lymphocytes (TIL) to cancer patients. TIL cells are more potent at killing tumors than LAK cells in animal experiments, but are difficult and expensive to generate for treatment of patients. TIL cells are autologous effector cells differentiated in vivo in solid tumors [see, U.S. Pat. No. 5,126,132, which describes a method for generating TIL cells for adoptive immunotherapy of cancer]. TIL cells are produced by removing a tumor sample from a patient, isolating lymphocytes that were infiltrating into the tumor sample, growing these TIL cells ex vivo in the presence of IL-2 and reinfusing the cells to the patient along with IL-2. A 60% response rate in evaluable cancer patients using this protocol has been reported [see, Rosenberg, et al. (1988) N. Engl. J. Med. 319:1676]. Another study reported a 23% response rate [see, Dillman, et al (1991) Cancer 68:1]. It, however, has been difficult to consistently propagate sufficient numbers of TIL cells for use in adoptive immunotherapy protocols.

In addition, the type of immune cells derived from TIL cultures are extremely variable. The cells recovered from tumor samples contain pure or mixed populations of cells with differing activities and potencies. Some cells are produced with MHC-restricted anti-tumor cytolytic activity, some with non-MHC restricted anti-tumor cytolytic activity and some without any anti-tumorcytolytic activity. Also, other than cultures derived from melanomas, cultures of TIL cells rarely produce tumor-specific cells from patients with solid tumors; and tumor-specific cells are produced only from about 50-75% of patients with metastatic melanoma.

Because TIL cell therapy is associated with extreme toxicity associated with infusion of IL-2, efforts have been made to enhance the efficacy of the treatment. For example, addition of IL-10 with IL-2 has been shown to increase the anti-tumor function of TIL cells in mice [see, Yang, et al. (1995) J. Immunol. 155:3897. Increasing the IL-6 concentration at the tumor site has also been shown to result in enhanced anti-tumor activity in TIL cells from mice [see, Marcus, et al. (1994) J. Immunoth. Emphasis Tumor Immunol. 15:105]. The anti-tumor activity of TIL cells is also increased by activating tumor draining lymph node cells with anti-CD3 mAb in the presence of IL-1 [see, Hammel, et al. (1994) J. Immunoth. Emphasis Tumor Immunol. 16:1].

Because of the variability in the effector function of cells derived from tumor infiltrates or draining lymph nodes, effort is being invested in development of methods to promote the ex vivo sensitization of tumor-reactive immune cells for use in adoptive immunotherapy of cancer. Tumor-antigen specific, MHC-restricted CTL from precursor cells present in the cellular infiltrates of breast cancer patients have been produced by incubating precursor cells with recombinant avipox MAGE-1 [a marker present on a class of tumors], causing the formation of MAGE-1 specific CTL [(MAGE-1 and other MAGE antigens are antigens expressed on tumor cells); see Toso, et al. (1996) Cancer Research 56:16; see, also U.S. Pat. No. 5,512,444]. Another ex vivo sensitization method for generating potent MHC-restricted CTL involves the incubation of peripheral blood mononuclear cells (PBMC) from melanoma patients with autologous, irradiated PBMC that have been pulsed with synthetic peptides of gp100, a melanoma-associated antigen [see, Salgaller, et al. (1995) Cancer Research 55:4972].

An alternative to TIL cells in adoptive immunotherapy of cancer are “ALT” cells. These cells are ex vivo activated peripheral blood lymphocytes with CTL activity. They are activated in an IL-2-containing supernatant derived from a previously prepared one-way mixed lymphocyte culture or by using cytokine-rich, autologous supernatant harvested from a previous lymphocyte culture stimulated with anti-CD3 mAb. Monthly infusions of ALT cells, combined with daily oral cimetidine (to reduce tumor-associated suppressor activity), significantly prolongs survival and induces durable tumor responses in renal cell carcinoma and melanoma patients [see, Graham, et al. (1993) Semin. Urol. 11:27 and Gold, et al. (1996) J. Surg. Res. 59:279].

Other effector immune cells have been used or proposed for adoptive immunotherapy of cancer. For example, the PWM-AK cell has been proposed as a possible candidate for adoptive immunotherapy of cancer. These effector cells are pokeweed mitogen activated PBMC with similar activity to LAK cells [see, Ohno, et al. (1994) Int. J. Immunopharm. 16:761]. Human activated macrophages (MAK) have also been proposed as effector cells in adoptive immunotherapy of cancer. The MAK cells are differentiated from the peripheral blood by activation with interferon-γ (IFN-γ) and have been shown to cause regression of experimental tumors in animals, but have not shown a clear therapeutic response in humans [see, Bartholeyns et al. (1994) Anticancer Research 14:2673]. Activated natural killer cells (ANK) have also been proposed for use in adoptive immunotherapy of malignancies. ANK cells are prepared by panning of peripheral blood stem cells on CD5/CD8 coated flasks yielding a population enriched for monocytes or NK precursors and then treating the cells with high concentrations of IL-2. A human-derived, MHC non-restricted CTL clone (TALL-104) has also shown promise for use in adoptive immunotherapy protocols for cancer treatment when used in conjunction with IL-12 [see, Cesano, et al (1994) J. Clin. Invest. 94:1076]. Increasing interest in the use of MAK, ANK and other mononuclear phagocytes in adoptive immunotherapy protocols for treatment of cancer has led to the development of improved methods to reproducibly harvest large numbers of functional human circulating blood monocytes by counterflow centrifugal elutriation [see, Faradiji, et al. (1994) J. Immunol. Methods 174:297].

An emerging adoptive immunotherapy strategy for treatment of cancer is to isolate and/or generate antigen presenting cells such as dendritic cells from a patient's blood, pulse the cells with tumor fragments or antigenic peptides and then reintroduce the cells to the patient [see, Grabbe, et al. (1995) Immunol. Today 16:117]. Methods for obtaining large numbers of dendritic cells from precursors in the blood of adults have been described [see, Romani, et al. (1994) J. Exp. Med. 180:83 and Bernhard, et al. (1995) Cancer Res. 55:1099].

Adoptive Immunotherapy and the Use Thereof for Treating Viral Diseases

Another application of immune cell adoptive immunotherapy is the treatment of viral disease. Adoptive immunotherapy protocols using viral-specific CD8+ and CD4+ effector cells have been developed for the treatment of infections with CMV, EBV and HIV [see, Riddell et al. (1995) Ann. Rev. Immunol. 13:545; van Lunzen, et al. (1995) Adv. Exp. Med. Biol. 374:57; and Klimas, et al. (1994) AIDS 8:1073]. These protocols involve purifying CD8+ T-cells from the peripheral blood of AIDS patients, expanding the cells with phytohemagglutinin and IL-2 and reinfusing the cells, with concomitant IL-2 infusion, to the patient [see, Whiteside, et al. (1993) Blood 81 :2085; Klimas, et al. (1994) AIDS 8:1073; Riddell, et al. (1993) Curr. Opin. Immunol. 5:484; Torpey, et al. (1993) Clin. Immunol. lmmunopath. 68:263;Ho, et al. (1993) Blood 81:2093 and Riddell, et al. (1992) Science 257:238].

Methods for Growing Immune Cells in vitro

A majority of adoptive immunotherapy protocols are hampered by the inability to grow clinically relevant (i.e., therapeutically sufficient) quantities of cells for infusion. An additional problem is that the administration of high doses of IL-2 necessary to maintain LAK activity and CTL activity in vivo is associated with severe toxicity. Several techniques have been reported for improving the growth of cells for adoptive immunotherapy and for reducing the dosage requirement for systemic administration of IL-2. None of these attempts to increase activity provided a means to eliminate IL-2 from the protocol.

TIL cells activated with anti-CD3 mAb and expanded with moderate amounts of IL-2 (100 U/ml) have been successfully used in adoptive immunotherapy protocols using less toxic systemic doses of IL-2 [see, Goedegebuure, et al. (1995) J. Clin. Oncol. 13:1939, see, also, Matsumura, et al. (1994) Cancer Research 54:2744]. In vivo administration of anti-CD3 mAb with low doses of IL-2 has also been suggested as an alternative adoptive immunotherapy strategy to lower the requirement for systemic IL-2 [see, Nakajima, et al. (1994) Proc. Natl. Acad. Sci. U.S.A. 91:7889]. A method for expanding CD4+ cells with helper and cytolytic function using immobilized anti-CD3 mAb and IL-2 in rotary-tissue culture bags has also been described [see, Nakamura, et al. (1993) Br. J. Cancer 67:865]. Co-culture of anti-tumor effector cells activated with anti-CD3 mAb with lipopolysaccharide (LPS)-activated B-cells has also been suggested as an alternative method for growing cells for adoptive immunotherapy [see, Okamoto, et al. (1995) Cancer Immunol. Immunoth. 40:173]. These cells are all subsequently expanded with low doses of IL-2.

A combination of mAbs against CD3 and CD28 in the presence of lower dose IL-2 induces efficient expansion of TIL cells [see, Mulder, et al. (1995) Cancer Immunol Immunoth. 41:293]. Anti-tumor CTL generated by in vitro stimulation with synthetic peptides can grow as long as 4 months in culture with low dose IL-2 (30 u/ml) [see, Salgaller, et al. (1995) Cancer Research 55:4972]. IL-7 has been shown to support the growth of CTL for prolonged periods in the absence of repeated stimulation [see, Lynch et al. (1994) J. Exp. Med. 179:31]. Low concentrations of IL-2 have also been used to grow TIL cells in artificial capillary culture systems [see, Freedman, et al. (1994) J. Immunoth. Emphasis Tumor Immunol. 16(3):198].

The need for exogenous IL-2 in expansion of immune cells has been obviated only by genetically modifying cells [see, e.g., U.S. Pat. No. 5,470,730]. All the methods for growing genetically unmodified cells, however, require exogenous IL-2 to promote the differentiation and/or growth of cells for use in adoptive immunotherapy protocols. All methods require systemic administration of IL-2 to maintain activity of such cells.

Despite the showing of efficacy of adoptive immunotherapy in terminally-ill patients, the severe toxicity of the systematic dosages of IL-2 required in adoptive immunotherapy protocols, the variability in the effector function of cell compositions derived from individual patients, as well as the difficulties in expanding clinically-relevant numbers of effector cells has limited the use of adoptive immunotherapy. In particular, the need for exogenous IL-2 limits the cells used in adoptive immunotherapy to effector cells that can perform their functions over a limited period of time. In order to exploit the potential of this treatment method, there is a need to overcome the need for systemic IL-2 administration, and the difficulties in obtaining large quantities of cells. Thus, there is a need for improved adoptive immunotherapy methods.

Therefore, it is an object herein to provide such improved methods. In particular, it is an object herein to provide methods for expanding immune cells for use in adoptive immunotherapy protocols without the use of exogenous IL-2. It is also an object herein to provide methods to generate a large array of cell compositions, including compositions containing regulatory cells, for use in adoptive immunotherapy protocols. It is an object herein to provide means to produce compositions containing clinically relevant numbers of such cells. he availability of a an array of cell compositions permits the design of adoptive immunotherapy protocols for a wide variety of diseases and immune function alterations. Therefore, it is an object herein to provide methods for treating various disorders and altering immune function.

### SUMMARY OF THE INVENTION

Compositions containing clinically relevant numbers of the immune cells are provided. The compositions contain regulatory immune cells, effector immune cells or combinations thereof. In particular compositions containing clinically relevant numbers of regulatory immune cells, especially Th1 and Th2 cells, for use in adoptive immunotherapy [herein referred to as autologous cell therapy (ACT)] are provided. Methods for generating the compositions containing the clinically relevant numbers of immune cells for use in adoptive immunotherapy are provided. The methods do not require use of IL-2. As a consequence, the expanded immune cells do not require IL-2 to retain activity or to remain viable.

Also provided are methods of treatment of disorders, including infectious diseases and autoimmune diseases. In addition, methods of treatment for immunosuppression permitting organ or tissue transplantation and methods for enhancement of vaccination protocols are provided. The treatment methods use the compositions.

The compositions of regulatory cells provide a means to alter the immunoregulatory balance of a patient, either locally or sytemically, by changing the predominant regulatory cell population. Because many disease states occur with the loss of regulated balance of the immune system that is normally maintained by regulatory immune cells, the availability of clinically-relevant numbers of regulatory immune cells provides a means to correct these imbalances. This ability offers great potential for treating a variety of diseases.

Methods for generating clinically relevant numbers of effector immune cells and of regulatory immune cells are provided. In particular, methods for generating substantially homogeneous populations of clinically relevant numbers of regulatory immune cells, including Th1 and Th2 cells, as well as Th1-like and Th2-like mononuclear cell populations are provided. Methods for generating compositions containing clinically relevant numbers of effector cells, such as CTLs, LAKS and TILS, that do not require exogenous IL-2 are provided.

Also provided are methods for producing clinically relevant quantities (i.e., therapeutically effective numbers, typically greater than 10⁹, preferably greater than 10¹⁰) of autologous specific T cell types for treatment of disease states where a relative deficiency of such cells is observed. In particular, methods for producing clinically relevant numbers of autologous, ex vivo derived Th1 T-cells from patients with disease states where a Th2 cytokine profile predominates such as, but not limited to, infectious and allergic diseases; and autologous, ex vivo derived Th2 T-cells in Th1-dominant diseases, such as, but not limited to, chronic inflammation and autoimmune diseases, for use in ACT protocols. The resulting cell compositions are provided and the use of the compositions in ACT protocols are provided.

Also provided are clinically relevant numbers of ex vivo derived antigen-specific Th2 cells sensitized to a donor organ for use in ACT protocols designed to provide specific immunosuppression for transplantation procedures. Clinically relevant numbers of ex vivo derived viral-specific Th1 cells for ACT protocols designed to provide protection from viral infection and thus serve as a viral vaccination strategy are also provided.

Methods of use of regulatory immune cells in autologous cell therapy (ACT) protocols to treat and prevent human disease are provided. The ACT protocols designed to alter the immunoregulatory balance of a patient in order to treat diseases where imbalances in regulatory cells exist. In particular, ACT protocols designed to alter the immunoregulatory balance of a patient in order to treat diseases where imbalances in regulatory cells exist are provided.

The methods involve collecting peripheral blood mononuclear cells from a patient and then expanding the cells by appropriate activation and then mitogenic stimulation with a cell surface specific proteins or proteins under conditions whereby clinically relevant numbers of the expanded cell type are produced [typically 10⁹, preferably 10¹⁰, more preferably 10¹¹, or more depending upon the cell type and ultimate application]. If the collected cells are not differentiated in vivo or require further differentiation, then following collection and prior to expansion, the method includes activating and causing differentiation of the cells ex vivo under conditions whereby at least some of the cells differentiate into regulatory or effector cells or other cell types. The resulting cells are then reinfused into the donor to effect treatment. The desired cells may be purified prior to reinfusion to provided a more homogeneous population.

Where required, differentiation of mononuclear cells is effected by activating the cells with a mitogen in the presence of the appropriate array of cytokines. This activation can be achieved by use of agents, such as cytokines or mitogens or other growth promoting agents under environmental conditions conducive to development of a particular phenotype. For example, if the cells are activated in the presence of IFN-γ, Th1 cell differentiation will be produced. If they are activated in the presence of IL-4, then Th2 cell differentiation will be produced. Such activating agents include monoclonal antibodies for polyclonal activation, and natural or synthetic antigens for specific activation presented in the context of MHC molecules.

Expansion is effected by growing the cells under conditions in which high cell densities can be achieved, whereby endogenous cytokines will be retained in the vicinity of the growing cell population, and in the presence of one or more mitogenic monoclonal antibodies or other cell surface specific protein, other than IL-2 or other such cytokine that will require co-infusion. Such conditions are preferably achieved by growing the cells in a hollow fiber [HF] bioreactor.

Methods for treating various disorders using the resulting cells are also provided. In effecting these methods, cells of a type that are found to be deficient or in low relative amounts are infused into a patient. For example, infectious diseases or tumors may be treated by collecting peripheral blood mononuclear cells from a patient; expanding the cells under conditions whereby a composition containing a therapeutically effective number of cells is produced; and infusing the resulting composition of cells into the patient. In preferred embodiments, the cells are specific for unique antigens in the vicinity of the site where an effect is desired or are specific for a pathogen or tumor being treated. In other preferred embodiments, effector cells, such as cytotoxic CD8⁺ T lymphocytes (CTLs) that are specific for the pathogen or tumor are infused or co-infused with regulatory cells.

In addition, methods for specific immunosuppression for transplantation procedures are provided. These methods involve administration of clinically relevant numbers of ex vivo derived antigen-specific Th2 cells sensitized to a donor organ. In preferred embodiments the cells are specific for alloantigens or an antigen unique to the organ or tissue being transplanted.

Also provided are vaccination methods and compositions for use as vaccines. In particular the vaccines are formulated from clinically relevant numbers of ex vivo-derived viral-specific Th1 cells or Th2 cells (or Th1-like or Th2-like populations of cells) that upon infusion provide protection from viral infection and thus serve as a viral vaccination strategy.

Methods of altering the immunoregulatory balance of a patient by infusing autologous, ex vivo derived and expanded regulatory immune cells are provided. This method includes the steps of collecting peripheral blood mononuclear cells from a patient, activating the cells ex vivo under conditions whereby at least some, even one, of the cells differentiate into the desired regulatory cells, expanding the regulatory cells, and infusing the expanded regulatory cells into the donor to affect the immunoregulatory balance. In particular, the infusion is not accompanied by co-infusion of a cytokine, such as IL-2.

The method above is useful for therapeutic treatment of disorders characterized by imbalances in regulatory immune cells. Specifically, the methods provided herein can be used to develop treatments for chronic inflammation in disorders such as, but not limited to, multiple sclerosis, rheumatoid arthritis, Crohn's Disease, autoimmune thyroid disease and inflammatory bowel disease; chronic infectious diseases such as infections with human immunodeficiency virus, herpes simplex virus, cytomegalovirus and hepatovirus; allergic and other hypersensitivity disorders such as asthma; and provides a method for specific immunosuppression in organ and tissue transplant procedures and a method to provide immunoprotection in vaccination.

In preferred embodiments, the regulatory immune cells are either Th1, Th2 or Th3 cells with a CD4⁺ or CD8⁺ phenotype. The cells will preferably have a “memory” phenotype (i.e., CD45RO⁺, L-selectin⁻), which permit the cells to traffic to sites of inflammation. These cells are preferably made to exert their regulatory function at a localized area of the body by selectively expanding cells specific for an unique antigen present at the site the regulatory effect of the cells is desired. For example, in the treatment of rheumatoid arthritis, regulatory cells specific for type II collagen, which is present only in joint tissue, are preferred. In the treatment of diabetes for preventing rejection of transplanted islet cells, regulatory cells specific for insulin are preferred.

In other embodiments, the cells are effector cells that have been expanded up to clinically relevant (i.e., therapeutically effective) numbers without the use of IL-2 to promote expansion.

Also provided is a method for expanding immune cells without the use of exogenous IL-2. The expansion of immune cells is preferably caused by the inclusion of one or more mitogenic mAb in the culture medium. The immune cells are preferably expanded under conditions in which they grow to high density. Such high density can be achieved by growing the cells in hollow fiber bioreactors with the molecular weight cut-offs of the fibers that retain endogenously produced cytokines. Such molecular weigh cut-off is preferably less than 14,000 daltons, more preferably 6000 daltons.

Also provided are methods for producing clinically relevant populations of virally purged CD4⁺ cells obtained from HIV⁺ patients. The resulting virally purged CD4⁺ cells are then reinfused into the donor patient in order to effect treatment of HIV. The cells may also be co-infused with anti-HIV effector cells.

### DETAILED DESCRIPTION OF THE PREFERRED EMBODIMENTS

A. Definitions

Unless defined otherwise, all technical and scientific terms used herein have the same meaning as is commonly understood by one of skill in the art to which this invention belongs. All patents and publications referred to herein are, unless noted otherwise, incorporated by reference in their entirety.

As used herein, adoptive immunotherapy or cellular adoptive immunotherapy refers to a method of treatment involving administration of immunologically active cells. The cells used in the treatment are generally obtained by venipuncture or leukopheresis either from the individual to be treated (autologous treatment) or from another individual (allogeneic). For purposes herein, autologous treatment is herein referred to as autologous cell therapy (ACT).

As used herein, autologous cell therapy [ACT] is a therapeutic method in which cells of the immune system are removed from an individual, cultured and/or manipulated ex vivo or in vitro, and introduced into the same individual as part of a therapeutic treatment.

As used herein, activating proteins are molecules that when contacted with a T-cell population cause the cells to proliferate. T-cells generally require two signals to proliferate. Activating proteins thus encompasses the combination of proteins that provide the requisite signals, which include an initial priming signal and a second co-stimulatory signal. The first signal requires a single agent, such as anti-CD3 mAb, anti-CD2 mAb, anti-TCR mAb, PHA, PMA, and other such signals. The second signal requires one or more agents, such as anti-CD28, anti-CD40L, cytokines and other such signals. Thus activating proteins include combinations of molecules including, but are not limited to: cell surface protein specific monoclonal antibodies, fusion proteins containing ligands for a cell surface protein, ligands for such cell surface proteins, or any molecule that specifically interacts with a cell surface receptor on a mononuclear cell and indirectly or directly causes that cell to proliferate. For purposes herein, when expanding effector cells, the activating proteins are selected from among those that are not needed to substantially maintain cell viability and function after expansion. Thus, for example, IL-2 is not an activating protein for purposes herein for effector cell expansion. As noted, the methods herein provide a means to produce cells, particularly effector, that do not require IL-2, and thus, in preferred embodiments, IL-2 will not be used as an activating agent.

As used herein, a mitogenic monoclonal antibody is an activating protein that is an antibody that when contacted with a cell directly or indirectly provides one of the two requisite signals for T-cell mitogenesis. Generally such antibodies will specifically bind to a cell surface receptor thereby inducing signal transduction that leads to cell proliferation. Suitable mitogenic antibodies may be identified empirically by testing selected antibodies singly or in combination for the ability to increase numbers of a specific effector cell. Suitable mitogenic antibodies or combinations thereof will increase the number of cells in a selected time period, typically 1 to 10 days, by at least about 50%, preferably about 100% and more preferably 150-200% or more, compared to the numbers of cells in the absence of the antibody.

As used herein, a growth promoting substance is a substance, that may be soluble or insoluble, that in some manner participates in or induces cells to differentiate, activate, grow and/or divide. Growth promoting substances include mitogens and cytokines. Examples of growth promoting substances include the fibroblast growth factors, osteogenin, which has been purified from demineralized bone [see, em., Luyten, et al (1989) J. Biol. Chem. 264:13377]), epidermal growth factor, the products of oncogenes, the interleukins, colony stimulating factors, and any other of such factors that are known to those of skill in the art. Recombinantly-produced growth promoting substances, such as recombinantly-produced interleukins, are suitable for use in the methods herein. Means to clone DNA encoding such proteins and the means to produce biologically active proteins from such cloned DNA are within the skill in the art. For example, interleukins 1 through 6 and others have been cloned. Various growth promoting substances and combinations thereof may be used to expand desired subpopulations of lymphoid cells.

As used herein, a mitogen is a substance that induces cells to divide and in particular, as used herein, are substances that stimulate a lymphocyte population in an antigen-independent manner to proliferate and differentiate into effector cells or regulatory cells. Examples of such substances include lectins and lipopolysaccharides.

As used herein, a cytokine is a factor, such as lymphokine or monokine, that is produced by cells that affect the same or other cells.

As used herein, a lymphokine is a substance that is produced and secreted by activated T lymphocytes and that affects the same or other cell types. Tumor necrosis factor, the interleukins and the interferons are examples of lymphokines. A monokine is a substance that is secreted by monocytes or macrophages that affects the same or other cells.

As used herein, a regulatory immune cell is any mononuclear cell with a defined cytokine production profile and in which such cytokine profile does not directly mediate an effector function. A regulatory immune cell is a mononuclear cell that has the ability to control or direct an immune response, but does not act as an effector cell in the response. Regulatory immune cells exert their regulatory function by virtue of the cytokines they produce and can be classified by virtue of their cytokine production profile. For example, regulatory immune cells that produce IL-2 and IFN-γ, but do not produce IL-4 are termed “Th1” cells. Regulatory immune cells that produce IL-4 and IL-10, but do not produce IFN-γ are termed “Th2” cells. Regulatory immune cells that produce TGF-β, IL-10 and IFN-γ, but do not produce IL-2 or IL-4 are termed “Th3” cells. Cells that produce Th1, Th2 and Th3 cytokine profiles occur in CD4+ and CD8+ cell populations. Cells that produce IL-2, IL-4 and IFN-γ are thought to be precursors of Th1 and Th2 cells and are designated “Th0” cells. Populations of cells that produce a majority of Th1 cytokines are designated “Th1-like”; populations producing a majority of the Th2 cytokines are designated Th2-like”; those producing a majority of Th3 cytokines are designated “Th3-like”. Thus, each composition, although containing a heterogeneous population of cells, will have the properties that are substantially similar, with respect to cytokine, to the particular Th subset.

It is understood that this list of T-cells is exemplary only, and any other definable population, array or subtype of T cells that can be expanded by the methods herein to clinically relevant numbers are intended herein.

As used herein, a composition containing a clinically relevant number or population of immune cells is a composition that contains at least 10⁹, preferably greater than 10⁹, more preferably at least 10¹⁰ cells, and most preferably more than 10¹⁰ cells, in which the majority of the cells have a defined regulatory or effector function, such as Th1 cells or Th2 cells or effector cells, such as LAK, TIL and CTL cells. The preferred number of cells will depend upon the ultimate use for which the composition is intended as will the type of cell. For example, if Th1 cells that are specific for a particular antigen are desired, then the population will contain greater than 50%, preferably greater than 70%, more preferably greater than 80%, most preferably greater than 90-95% of such cells. If the population results from polyclonal expansion, the homogeneous cells will be those that are a particular type or subtype. For uses provided herein, the cells are preferably in a volume of a liter or less, more preferably 500 mls or less, even more preferably 250 mls or less and most preferably about 100 mls or less.

As used herein, predominant means greater than about 50%.

As used herein, a combination refers to two component items, such as compositions or mixtures, that are intended for use either together or sequentially. The combination may be provided as a mixture of the components or as separate components packaged or provided together, such as in a kit.

As used herein, effector cells are mononuclear cells that have the ability to directly eliminate pathogens or tumor cells. Such cells include, but are not limited to, LAK cells, MAK cells and other mononuclear phagocytes, TILs, CTLs and antibody-producing B cells and other such cells.

As used herein, immune balance refers to the normal ratios, and absolute numbers, of various immune cells that are associated with a disease free state. Restoration of immune balance refers to restoration to a condition in which treatment of the disease or disorder is effected whereby the ratios of regulatory immune cell types and numbers thereof are within normal range or close enough thereto so that symptoms of the treated disease or disorder are ameliorated. The amount of cells to administer can be determined empirically, or, preferably, by administering aliquots of cells to a patient until the symptoms of the disease or disorder are reduced or eliminated. Generally a first dosage will be at least 10⁹-10¹⁰ cells. In addition, the dosage will vary depending upon treatment sought. As intended herein, about 10⁹ is from about 5×10⁸ up to about 5×10⁹; similarly about 10¹⁰ is from about 5×10⁹ up to about 5×10¹⁰, and so on for each order of magnitude.

As used herein, therapeutically effective refers to an amount of cells that is sufficient to ameliorate, or in some manner reduce the symptoms associated with a disease. When used with reference to a method, the method is sufficiently effective to ameliorate, or in some manner reduce the symptoms associated with a disease.

As used herein, mononuclear or lymphoid cells (the terms are used interchangeably) include lymphocytes, macrophages, and monocytes that are derived from any tissue in which such cells are present. In general lymphoid cells are removed from an individual who is to be treated. The lymphoid cells may be derived from a tumor, peripheral blood, or other tissues, such as the lymph nodes and spleen that contain or produce lymphoid cells.

As used herein, therapeutically useful subpopulations of in vitro or ex vivo expanded mononuclear or lymphoid cells are cells that are expanded upon exposure of the cells to a growth promoting substances, such as lymphokines, when the lymphoid cells are cultured ex vivo. The therapeutically useful subpopulations are regulatory cells or effector cells and contain clinically relevant numbers of cells, typically at least about 10⁹ or more cells, which are preferably in a clinically useful volume (i.e., for infusion) that is one liter or less.

As used herein, a therapeutically effective number or clinically-relevant number ex vivo expanded cells is the number of such cells that is at least sufficient to achieve a desired therapeutic effect, when such cells are used in a particular method of ACT. Typically such number is at least 10⁹, and more preferably 10¹⁰ or more. The precise number will depend upon the cell type and also the intended target or result.

As used herein, a hollow fiber bioreactor or hollow fiber bioreactor cartridge contains an outer shell casing that is suitable for the growth of mammalian cells, a plurality of semi-permeable hollow fibers encased within the shell that are suitable for the growth of mammalian cells on or near them, and the ECS, which contains the cells and the ECS cell supernatant. The interior of the hollow fibers is called the lumen and the area between the outside of the capillaries to the inside of the outer housing is called the extracapillary space [ECS].

Tissue culture medium perfuses through the fiber lumens and is also included within the shell surrounding said fibers. The tissue culture medium, which may differ in these two compartments, contains diffusible components that are capable of sustaining and permitting proliferation of immune cells. The medium is provided in a reservoir from which it is pumped through the fibers. The flow rate can be controlled varied by the varying the applied pressure. The ECS or perfusing medium may additionally contain an effective amount of at least one growth promoting or suppressing substance that specifically promotes the expansion or suppression of at least one subpopulation of the immune cells, such as TIL cells or regulatory cells, in which the effective amount is an amount sufficient to effect said specific expansion.

As used herein, a hollow cell fiber culture system includes of a hollow fiber bioreactor as well as pumping means for perfusing medium through said system, reservoir means for providing and collecting medium, and other components, including electronic controlling, recording or sensing devices. A hollow fiber bioreactor is a cartridge that contains of a multitude of semi-permeable tube-shaped fibers encased in a hollow shell. The terms hollow fiber reactor and hollow fiber bioreactor are used interchangeably. A preferred device for methods is that described in copending, allowed, U.S. application Ser. No. 08/506,173.

As used herein, ECS refers to the extra-capillary space cell supernatant. It is the medium in which the cells in the ECS are growing. It contains secreted cellular products, diffusible nutrients and any growth promoting or suppressing substances, such as lymphokines and cytokines, produced by the cultured immune cells or added to the ECS or tissue culture medium. The particular components included in the ECS is a function not only of what is inoculated therein, but also of the characteristics of the selected hollow fiber.

As used herein, tissue culture medium includes any culture medium that is suitable for the growth of mammalian cells ex vivo. Examples of such medium include, but are not limited to AIM-V, RPMI 1640, and Iscove's medium (GIBCO, Grand Island, N.Y.). The medium may be supplemented with additional ingredients including serum, serum proteins, growth suppressing, and growth promoting substances, such mitogenic monoclonal antibodies and selective agents for selecting genetically engineered or modified cells.

As used herein, treatment means any manner in which the symptoms of a condition, disorder or disease are ameliorated or otherwise beneficially altered. Treatment also encompasses any pharmaceutical use of the compositions herein.

As used herein, a vaccine is a composition that provides protection against a viral infection, cancer or other disorder or treatment for a viral infection, cancer or other disorder. Protection against a viral infection, cancer or other disorder will either completely prevent infection or the tumor or other disorder or will reduce the severity or duration of infection, tumor or other disorder if subsequently infected or afflicted with the disorder. Treatment will cause an amelioration in one or more symptoms or a decrease in severity or duration.

As used herein, amelioration of the symptoms of a particular disorder by administration of a particular composition refers to any lessening, whether permanent or temporary, lasting or transient that can be attributed to or associated with administration of the composition.

As used herein, substantially pure means sufficiently homogeneous to appear free of readily detectable impurities as determined by standard methods of analysis, such as flow cytometry, used by those of skill in the art to assess such purity, or sufficiently pure such that further purification would not detectably alter the physical and chemical properties, such as biological activities, of the substance. Methods for purification of the immune cells to produce substantially pure populations are known to those of skill in the art. A substantially pure cell population, may, however, be a mixture of subtypes; purity refers to the activity profile of the population. In such instances, further purification might increase the specific activity of the cell population.

As used herein, biological activity refers to the in vivo activities of immune cells or physiological responses that result upon in vivo administration of a cell, composition or other mixture. Biological activity, thus, encompasses therapeutic effects and pharmaceutical activity of such cells, compositions and mixtures.

Although any similar or equivalent methods and materials can be employed in the practice and/or tests of the methods and cells provided herein, preferred embodiments are now described.

B. Effector and Regulatory Immune Cells

Encounter of a host with antigen can result in either cell-mediated or humoral classes of immune response. Regulatory immune cells control the nature of an immune response to pathogens [see, Mosmann, et al. (1986) J. Immunol. 136:2348; Cherwinski, et al. (1987) J. Exp. Med. 166:1229; and Del Prete, et al. (1991) J. Clin. Invest. 88:346]. The different types of responses are attributable to the heterogeneity of CD4+ T cells. CD4+ cells can be sub-divided according to their cytokine expression profiles. These cells are derived from a common precursor, Th0, which can produce Th1, Th2 and Th3 cytokines [see, Firestein, et al. (1989) J. Immunol. 143:518]. As noted above, Th1 clones produce IL-2, INF-γ, lymphotoxin and other factors responsible for promoting delayed-type hypersensitivity reactions characteristic of cell-mediated immunity. These cells do not express IL-4 or IL-5. Th1 cells promote cell-mediated inflammatory reactions, support macrophage activation, immunoglobulin (Ig) isotype switching to IgG2a and activate cytotoxic function.

Th2 clones produce cytokines, such as IL-4, II-5, IL-6, IL-10 and IL-13, and thus direct humoral immune responses, and also promote allergic type responses. Th2 cells do not express IL-2 and IFN-γ. Th2 cells provide help for B-cell activation, for switching to the IgG1 and IgE isotypes and for antibody production [see, em., Mosmann et al. (1989) Annu. Rev. Immunol. 7:145]. Th3 cell produce IL-4, IL-10 and TGF-β.

The cytokines produced by Th1 and Th2 cells are mutually inhibitory. Th1 cytokines inhibit the proliferation of Th2 cells and Th2 cytokines inhibit Th1 cytokine synthesis [see, e.g., Fiorentino, et al. (1989) Med. 170:2081 (1989). This cross regulation results in a polarized Th1 or Th2 immune response to pathogens that can result in host resistance or susceptibility to infection.

Development of the appropriate regulatory immune cell response during infection is important because certain pathogens are most effectively controlled by either a predominantly Th1 or Th2 type immune response [see, e.g., Sher, et al. (1989) Ann. Rev. Immunol. 46:111; Scott, et al. (1991) Immunol. Today 12:346; Sher, et al. (1992) Immunol. Rev. 127:183; and Urban, et al. (1992) Immunol. Rev. 127:205]. For example, a correlation has been found between the predominant regulatory immune response and disease susceptibility in leprosy [see, e.g., Yamamura, et al. (1991) Science 254:277] AIDS [see, e.g., Clerici, et al. (1993) Immunol. Today 14:107], toxoplasma [see, Sher, et al. (1989) Ann. Rev. Immunol. 46:111], Hashimoto's thyroiditis [see, e.g., Del Prete, et al. (1989) Autoimmunity 4:267], Grave's disease [see, e.g., Turner, et al. (1987) Eur. J. Immunol. 17:1807], transplantation [see, e.g., Benvenuto, et al. (1991) Transplantation 51:887], type 1 diabetes [see, e.g., Foulig, et al. (1991) J. Pathol. 165:97], multiple sclerosis [see, e.g., Benvenuto, et al. (1991) Clin. Exp. Immunol. 84:97], and rheumatoid arthritis [see, e.g, Quayle, et al. (1993) Scand. J. Immunol 38:75].

A Th1 response in mice to protozoan, viral and fungal infection is associated with resistance, while a Th2 response is associated with disease. A Th2 response cures certain helminth infections in mice and exacerbates viral infections. A Th2 response has been correlated with AIDS and autoimmune disease in humans and with allergic disorders and transplant rejection. Another regulatory cell, designated Th3, produces high amounts of TGF-β and can protect mice from a disease similar to multiple sclerosis [see, em., Chen, et al. (1994) Science 265:1237]. Categorization of these responses may be empirically determined and have been documented [for a summary see, e.g., Mosmann et al. (1996) Immunology Today 17:138-146].

Subsets of CD8⁺ T-cells also are known to secrete a Th1- or Th2-cytokine pattern. Exposure of CD8⁺ cells to IFN-γ and IL-2 direct differentiation into Th1 cells; whereas, IL-4 induces differentiation into Th2 cells. Th1 CD8⁺ cells are thought to be important effectors in the immune response to viruses, while Th2 CD8⁺ cells have an immunosuppressive function. Other regulatory cells can be characterized by methods similar to those used to characterize the above-described cells.

By virtue of the cross regulation and the immune imbalances observed in disease states, as described herein, regulatory cells should be therapeutic for the treatment of a variety of diseases. Such use has been demonstrated to some extent in animal models, but has not been possible to achieve in humans. For example, administration of native T-cells and Th2 antigen-specific clones for Actinobacillus actinomycetemcomitans, in combination did ameliorate periodontal disease in nude rats [see, Eastcott, et al. (1994) Oral Microbiol. Immunol. 9:284 (1994)]. Antigen-specific Th1 cell clones have been shown to protect against infection with the protozoan Leishmania major, genital infection with chlamydia trachomatis and murine candidiasis [see, Powrie, et al. (1994) J. Exp. Med. 179:589; Igietseme, et al. (1993) et al. Regional Immunity 5:317; and Romani (1991) Inf. Immun. 59:4647]. In addition, Th2 cell clones have been shown to prevent autoimmune uveoretinitis [see Saoudi, et al. (1993) Eur. J. Immunol. 23:3096]. An antigen-specific Th2 cell clone has been shown to suppress an animal model of multiple sclerosis [see, Chen, et al. (1994) Science 265:1237]. Donor-specific Th2 cells can reduce lethal graft vs. host disease in transplantation [see, Fowler, et al. (1994) Adv. Bone Marrow Purg. Process., Fourth Int. Sympos., Wiley-Liss, Inc., p. 533]. Purified T-cells with enhanced Th2 activity have also been shown to prevent insulin-dependent diabetes-like disease in animals. See, Fowell et al. (1993) J. Exp. Med. 177:627.

While Th2 clones have been used in adoptive transfer studies in animals, regulatory cells, including Th1 and Th2 cells, have not been used in ACT protocols in humans. Such protocols are limited by the inability to differentiate and produce therapeutically effective quantities of such regulatory cells. The methods herein, however, provide a means to produce such clinically relevant quantities of cells, and, thereby provide a means to ameliorate disorders, provide vaccines, and suppress tissue or organ rejection. The methods herein also provide a means to produce clinically relevant quantities of relulatory and effector cells in the absence of IL-2.

Also provided herein, are methods for growing cells that are therapeutically useful for treatment of HIV infection, including treatment of A.I.D.S. by enchancing or restoring the immune system [see, em., Examples 3 and 4].

C. Methods for Production of Regulatory Cells

A method for obtaining regulatory cells for use in ACT protocols is provided herein. A method for obtaining effector cells for use in ACT protocols without the need for exogenous agents, such as IL-2, that sustain the viability of such cells is also provided. The method includes some or all of the following steps: (1) collecting mononuclear cells from a patient; (2) treating the cells ex vivo with that agents that cause some or all of the cells to the differentiate into desired T cell subtypes; (3) purifying the resulting cells; and (4) expanding these cells by contacting them with a mitogenic agent that specifically interacts with a cell surface receptor. Such agents are herein preferably mitogenic monoclonal antibodies. The expanded cells may be further purified to select for the desired subtype.

1. Collecting Mononuclear Cells

Mononuclear cells (i.e., lymphocytes and monocytes) can be obtained from a variety of sources, including, but not limited to, peripheral blood, lymphoid tissue, biopsy tissue or from body cavity lavage procedures. Preferably, the cells are obtained by simple venipuncture (50-500 ml). When larger numbers of cells are required, they may be obtained by a lymphapheresis procedure. The mononuclear cells can be purified from the blood using Ficoll-Hypaque density gradient centrifugation or any other suitable method.

a. Ex Vivo Differentiation

Many studies have indicated that different antigens can cause a selective induction of distinct immunoregulatory cell subsets, causing the development of either a humoral or cell-mediated immune response. Furthermore, many disease states are the result of the predominance of the certain cell types. Recent advances in the understanding of the mechanisms regulating the differentiation of T-cell subsets allows the generation of selected subsets ex vivo.

Several factors, including the dose of antigen, the type of antigen presenting cell and the MHC haplotype of an individual can affect the differentiation of specific types of regulatory immune cells. Various cytokines are also able to affect the type of regulatory response that develops in a person. For example, it is known that the presence of IL-4 during initial T-cell activation gives rise to Th2-like cells [see, Hsieh, et al. (1992) Proc. Natl. Acad. Sci. U.S.A. 89:6065 and Paliard, et al. (1988) et al. J. Immunol. 141:849]. Conversely, activation of cells in the presence of IL-12 or interferon-gamma leads to the formation of Th1-like cells [see, Sedar, et al. (1993) Proc. Nati. Acad. Sci. U.S.A. 90:10188].

Accordingly, in a preferred embodiment, the mononuclear cells collected in the first step of the present process are next activated in the presence of IL-12, interferon-gamma or IL-4 to cause the development of Th1 or Th2 cells, respectively. To enhance the differentiation of regulatory cells, antibodies to IL-12 and/or interferon-gamma can be used to promote Th2 responses, while antibodies to IL-4 can be used to promote the differentiation of Th1 cells. Antibodies or other proteins specific for the IL-12, interferon-gamma or IL-4 receptor on T-cells could also be used to provide a signal in place of the lymphokines. The cells can be activated either non-specifically with chemical agents such as PHA and PMA or with monoclonal antibodies such as anti-CD3 or anti-CD2. Preferably, they are activated specifically with natural or man-made protein antigens added to the medium, processed and presented by APC to T-cells. It may be necessary in some cases to vaccinate the patient prior to blood collection in order to increase the starting number of antigen-specific cells. Another strategy is to oral tolerize patients prior to blood collection. In cases where the cells generated are specific for a known antigen, the antigen may also be used after the cell reinfusion as a booster to increase the desired regulatory cells In vivo. Additional strategies for effecting Th1 cell differentiation is to activate cells in the presence of αB7.2 mAb or TGF-β. Th2 differentiation also can be promoted by activating cells in the presence of one or more of agents, such as, one or more of the following: αB7.1 mAb, low antigen doses and CTLA4/lg fusion protein (CTLA4 is a ligand for CD28). CD28 is expressed on T-cells and antigen presenting cells.

The type of regulatory cells generated should be determined from animal models of the disease. It is known that not all regulatory cells within a classification are alike. For example, some Th2 cells secrete high levels of IL-4 and low levels of IL-10, while others have increased levels of IL-5. Other regulatory cells produce IL-10 and interferon-gamma. Regulatory cells termed “Th3” cells secrete TGF-β and are deemed preferential for treatment of multiple sclerosis.

b. Regulatory Cell Isolation

Most techniques for isolation of immune cell subsets are based on the reactivity of mAb against T-cell surface antigens. Positive selection can be achieved by fluorescent-activated cell sorting [see, Reinherz, et al. (1979) Proc. Natl. Acad. Sci. U.S.A. 76:4061]. Various panning techniques where specific mAb are bound to plastic plates to capture the desired T-cell subsets can also be used. See, Lum, et al. (1982) Cell Immunol. 72:122.

Panning techniques can be used for negative selection as well, depleting unwanted subsets with specific mAb [see, e.g., Engleman, et al. (1981) J. Immunol. 127:2124]. The use of magnetic polymer beads coated with mAb is a preferred method to isolate highly purified, functionally intact lymphoid cell populations by positive and negative selection [see, em., Lea, et al. (1985) Scand. J. Immunol. 22:207; Lea, et al. (1986) Scand. J. Immunol. 23:509) and Gaudernack, et al. (1986) J. Immunol. Methods 90:179].

Since an antibody has not yet been described that can distinguish regulatory immune cell subsets, efforts must be made to enhance the desired population by purifying on the basis of certain cell surface proteins. For example, CD30 positive [see, Manetti, et al. (1994) J. Exp. Med. 180:2407], CD27 negative [see, Elson, et al. (1994) Int. Immunol. 6:1003] and CD7 negative [see, Autran, et al. (1995) J. Immunol. 154:1408] cell populations have been shown to have the majority of Th2 cells. Also, repeatedly contacting the cells with anti-CD28 mAb is another method for enhancing Th2 cells.

Another strategy for purification of regulatory cells is to expand the cells in the presence of agents known to inhibit the growth of the unwanted subset(s) of cell. Such agents include dexamethasone, colchicine, CTLA4/lg fusion protein and progesterone, which inhibit Th2 cell growth. TGF-β inhibits Th1 cell growth.

C. Regulatory Cell Expansion

Methods for expanding purified T-cells to clinically relevant numbers ex vivo without the use of exogenous IL-2 are provided herein. Although IL-2 could be used in the present methods, it is preferably to grow cells without the addition of this cytokine. Cells exposed to IL-2 ex vivo may become dependent on the presence of IL-2 to maintain their viability and function, requiring the systemic infusion of IL-2 with the cells to the patient. Because the systemic infusion of IL-2 is known to be extremely toxic to patients, it is best to avoid the necessity for this cytokine.

In order for T-cells to proliferate, they require two separate signals.

The first signal is generally delivered through the CD3/TCR antigen complex on the surface of the cells. The second is generally provided through the IL-2 receptor. In order to bypass the IL-2 signal, combinations of mAb are used. Preferably, the mAb are in the soluble phase or immobilized on plastic or magnetic beads, in order to simplify the cell harvesting procedure.

(i) First Signal

To provide the first signal, it is preferable to activate cells with mAb to the CD3/TCR complex, but other suitable signals, such as, but not limited to, antigens, super antigens, polyclonal activators, anti-CD2 and anti-TCR antibodies, may be used. Other suitable agents can be empirically identified. Immobilized or cross-linked anti-CD3 mAb, such as OKT3 or 64.1, can activate T-cells in a polyclonal manner [see, Tax, et al. (1983) Nature 304:445]. Other polyclonal activators, however, such as phorbol myristate acetate can also be used [see, eg., Hansen, et al. (1980) Immunogenetics 10:2471.

Monovalent anti-CD3 mAb in the soluble phase can also be used to activate T-cells [see, Tamura, et al. (1992) J. Immunol. 148:2370]. Stimulation of CD4+ cells with monovalent anti-CD3 mAb in the soluble form is preferable for expansion of Th2 cells, but not Th1 cells [see, deJong, et al. (1992) J. Immunol. 149:2795]. Soluble heteroconjugates of anti-CD3 and anti-T-cell surface antigen mAb can preferentially activate a particular T-cell subset [see, Ledbetter, et al. (1988) Eur. S. Immunol. 18:525]. Anti-CD2 mAb can also activate T-cells [see, Huet, et al. (1986) J. Immunol. 137:1420]. Anti-MHC class 11 mAb can have a synergistic effect with anti-CD3 in inducing T-cell proliferation [see, Spertini, et al (1992) J. Immunol. 149:65]. Anti-CD44 mAb can activate T-cells in a fashion similar to anti-CD3 mAb. See, Galandrini, et al. (1993) J. Immunol. 150:4225].

For purposes herein, monoclonal antibodies to anti-CD3 are preferred. Anti-CD3 is used because CD3 is adjacent to the T-cell receptor. Triggering of CD3, such as by monoclonal antibody interaction, causes concomitant T cell activation.

(ii) Second Signal

To then cause proliferation of such activated T cells, a second signal is required. A variety of mAb singly or in combination can provide the second signal for T-cell proliferation. Anti-IL-4R mAb (specific for the interleukin-4 receptor molecule) can enhance the proliferation of the Th2 cells [see, Lindquist, et al. (1993) J. Immunol. 150:394]. Immobilized ligands or mAb against CD4, CD8, CD11a (LFA-1), CD49 (VLA), CD45RO, CD44 and CD28 can also be used to enhance T-cell proliferation [see, Manger, et al. (1985) J. Immunol. 135:3669;Hara, et al. (1985) J. Exp. Med. 161:1513; Shimizu, et al. (1990) J. Immunol. 145:59; and Springer, (1990) Nature 346:425]. Cell surface proteins that are ligans to B-cells are preferred targets for Th2 cell proliferation, while macrophage ligands are preferred for Th1 cell proliferation.

Anti-CD28 mAb in combination with anti-CD3 or anti-CD2 induces a long lasting T-cell proliferative response [see, Pierres, et al. (1988) Eur. J. Immunol. 18:685]. Anti-CD28 mAb in combination with anti-CD5 mAb results in an enhanced proliferative response that can be sustained for weeks [see, Ledbetter, et al. (1985) J. Immunol. 135:2331]. Anti-CD5 mAb alone can also provide a second signal for T-cell proliferation [see, Vandenberghe et al. (1991) Eur. J. Immunol. 21:251]. Other mAb known to support T-cell proliferation include anti-CD45 and CD27 [see, Ledbetter, et al. (1985) J. Immunol. 135:1819 and Van Lier, et al. (1987) J. Immunol. 139:1589].

To determine the combination of mAbs or proteins that optimally induce sustained regulatory cell proliferation, a screening procedure using combinations of these mAbs or proteins is used. The cells are incubated with various combinations of these substances and screened for growth by analysis of ³H-thymidine incorporation or equivalent methods. The group demonstrating the best growth characteristics is selected for use in the medium.

(iii) Expansion

In order to expand purified T-cells to clinically relevant numbers of up to 100 billion (10¹¹), the cells should be grown to high density. This can be achieved using any suitable means, including, but not limited to: stirred tank fermentors, airlift fermentors, roller bottles, culture bags, and other bioreactor devices. Hollow fiber bioreactors are presently preferred. Hollow fiber bioreactors permit cells to be cultured to the required high densities in a minimal volume. This reduces the amount of monoclonal antibodies, serum and medium required in the production process. In addition, selection of fibers with molecular weight cut-offs of 6000 daltons will allow continuous feeding and waste product removal while retaining cell derived cytokines in the culture space. These cytokines, such as IL-2 and IL-4, promote and sustain cell viability and proliferation.

T-cells, like most mammalian cells, will grow to a maximum density of 1×10⁶ cells/ml in tissue culture. Thus, a total of 100 liters of culture medium would be required to support 100 billion cells. In addition, the 100 liters of medium would have to be replenished regularly to maintain a proper nutrient/waste product balance necessary to keep the cells viable. A method would also be required to keep the 100 liters of medium saturated with oxygen.

Hollow fiber technology for cell culture is well known [see, e.g., U.S. Pat. Nos. 4,220,725, 4,206,015, 4,200,689, 3,883,393, and 3,821,087; see, also, U.S. Pat. No. 4,391,912; U.S. Pat. No. 4,546,083; U.S. Pat. No. 4,301,249; U.S. Pat. No. 4,973,558, U.S. Pat. No. 4,999,298; and U.S. Pat. No. 4,629,686] and is used to achieve issue-like cell densities in culture [i.e. densities of greater than about 10⁸ cells/ml]. The original hollow fiber bioreactor contains a housing with a plurality of artificial capillary hollow fiber membranes. The capillaries extend between an inflow opening at one end of the device and an outflow opening at the other. The capillaries have selectively permeable walls though which dissolved medium components can diffuse. The lumen and ECS are separated by potting material at the inflow and outflow openings. The housing also contains ports for access to the ECS enabling cells to be inoculated into the ECS [see, e.g., U.S. Pat. Nos. 3,821,087; 3,883,393 and 4,220,725, 4,206,015, 4,200,689, 3,883,393, and 3,821,087; see, also Knazek, et al. (1972) Science 178:65].

Hollow fiber technology permits cells to grow to densities 100-fold greater than cell densities [1×10⁸ cells/ml or greater] observed in conventional cell culture. Thus, only one liter of culture volume is required to generate 100 billion cells. The reduced cell volume would also decrease the amount of human serum and soluble mAb required in the expansion process. In addition, high cell densities provide environments that are a closer approximation to in vivo condition. The hollow fiber bioreactor is a component of a hollow fiber cell culture system. A typical hollow fiber cell culture system, such as the CELLMAX™ 100 hollow fiber cell culture system (Cellco Advanced Bioreactors, Inc., MD) contains a standard glass medium bottle, which serves as the reservoir, stainless steel/Ryton gear pump, an autoclavable hollow fiber bioreactor, which contains the fibers and shell casing in which cells are cultured, and medical grade silicone rubber tubing, or other connecting means, which serves as a gas exchanger to maintain the appropriate pH and pO₂ of the culture medium. All components are secured to a stainless steel tray of sufficiently small dimensions to enable four such systems to fit within a standard tissue culture incubator chamber. The pump speed and automatic reversal of flow direction are determined by an electronic control unit which is placed outside of the incubator and is connected to the pump motor via a flat ribbon cable which passes through the gasket of the incubator door. The pump motor is magnetically coupled to the pump and is lifted from the system prior to steam autoclaving.

The preferred HF bioreactor system for use herein is described in copending, allowed, U.S. application Ser. No. 08/506,173.

2. Preferred Hollow Fiber System for Large Scale T-Cell Cultures

A HF system that closely emulates in vivo conditions thereby permitting T-cells to grow to densities of over 1×10⁷ cells/mls, preferably 1×10⁸ cells/ml, that uses fibers with a low molecular weight cutoff to retain mitogenic mAbs and serum components, and that does not have gradient formation problems, is described in copending, allowed, U.S. application Ser. No. 08/506,173. This HF device allows outflow of the lumenal flow to be completely blocked. This leads to equal perfusion of nutrients along the entire length of the hollow fiber capillaries. It also includes an oxygen feed on the ECS of the bioreactor to provide desired oxygen delivery characteristics.

Artificial kidney cartridges [CD Medical of Hialeah, FL] having a length of 14 inches, an ECS volume of volume of 120 ml, and a molecular weight cutoff (MWC) of 6,000 daltons were selected as the hollow fiber bioreactors for use in the hollow fiber processing apparatus. To ensure equal distribution of nutrients across the entire length of these low MWC cartridges, an automatic on/off solenoid valve was placed on the outflow opening of the bioreactor. When the solenoid is in the “off” position, medium is prevented from exiting the bioreactor. Instead, the medium ultrafiltrates to the cells in the ECS equally to all points of the bioreactor. The medium then passes out of the bioreactor through the ports. Ultrafiltration of nutrients is more physiological and therefore more desirable for maintenance of dense cultures of cells [see, em., Swaab et al. (1974) Cancer Res. 34:2814; and Davis et al. (1974) Chem. Eng. J. 7:213].

To remove the metabolic waste from the cells in the ECS, the solenoid valve is switched to the “on” position and the medium is returned at a controlled pressure to the ECS through the eist ports. The medium then moves radially into the lumen. Finally, the medium is carried out the outflow opening.

The hollow fiber system permits the medium that ultrafiltrates from the lumen to the ECS (Cycle 1) to be automatically replenished with oxygen and for the levels of glucose, lactate and carbon dioxide to be adjusted. This reconditioned medium is then returned to the ECS when the solenoid valve is opened in Cycle 2. The same adjustments are conducted for medium on the lumenal side of the bioreactor. In this manner, oxygen diffusion limitations can be overcome as oxygen is supplied to the lumen and the ECS of the bioreactor, eliminating diffusion across the hollow fiber capillaries as the sole means of oxygen transfer.

For large-scale growth of regulatory immune cells hollow fiber bioreactors that have improved fluid dynamics to reduce gradient formation are preferable [see, em., U.S. Pat. No. 4,804,628, see, especially, allowed copending U.S. application Ser. No. 08/506,173] are presently preferred. The hollow fiber bioreactors that have such improved fluid dynamics are best suited for the large-scale growth of regulatory immune cells.

In preferred embodiments, mitogenic monoclonal antibodies are coated onto the hollow fiber surafce in order to deliver the proper signals necessary to cause the immune cells to divide.

D. Effector Cell Expansion

Effector cells are mononuclear cells that have the ability to directly eliminate pathogens or tumor cells. Such cells include, LAK cells, TILs, CTLs and antibody-producing B cells and other such cells. These cells are produced by first treating cells collected from a patient in manner known to lead to differentiation of such cells. For example, TIL cells are produced by culturing solid tumor tissue obtained by biopsy in IL-2 and/or other agents that lead to TIL production. The cells are then activated and expanded in the presence of mitogenic agents, such as monoclonal antibodies specific for cell surface receptors or other agents, as described above for the regulatory cells.

In accord with the methods provided herein, the cells are not exposed to exogenous IL-2 (or any other agent upon which the cells will become dependent for in vivo activity or survival) and reinfusion is not accompanied by co-infusion of IL-2.

E. Selection of Immune Cell Phenotype

Depending on the site of action at which a regulatory effect of infused cells is required (or at which effector cells are required), different cell phenotypes may be required. Lymphocytes recirculate extensively throughout the body and then localize in tissues and lymphoid organs. This is accomplished by an array of adhesion molecules on lymphocytes and counter-receptors on the vascular endothelium, extracellular matrix and epithelium. Recent studies have identified several of the specific receptor/ligand interactions that mediate lymphocyte trafficking.

Infused cells that need to migrate out of circulation (e.g., to sites of inflammation) must have the capacity to move through extracellular matrix (ECM) of various compositions. For example, subendothelial basement membrane presents a barrier rich in type IV collagen, laminin and heparan sulfate proteoglycans. The ECM of the interstitium contains collagens I and III, as well as various glycosaminoglycans such as hyaluronic acid. Fibronectin and vitronectin are also encountered in basement membrane and interstitium. Immune cells can be loaded into columns containing these materials in order to screen for cells capable of migration through the interstitium.

It is also know that cells with a “memory” phenotype (i.e., CD45RA−, CD45RO+, CD29+, CD11a+, CD44+, CD54+, CD58+, L-selectin−) will accumulate non-specifically at sites of chronic inflammation. Cells that express L-selectin are least likely to migrate and should be used when the desired regulatory effect is required in the lymphatic organs.

Growing out cells with a defined antigen specificity may also be desired in order to prevent non-specific immunoregulation. Antigens should be selected that are unique to the site a regulatory effect is desired or to the disease-causing antigen(s).

F. Practice of the Therapeutic Methods

The therapeutic methods herein are designed to produce compositions containing clinically relevant [at least 10⁹, preferably 10¹⁰, cells or more] populations of regulatory immune cells and/or effector immune cells for autologous infusion for treatment. The methods herein do not rely or use any agents for expansion that must be present after expansion to maintain cell viability or activity. In particular, expansion does not require or use IL-2. As a result, re-infusion of the cells does not require or use IL-2, thereby obviating toxicity and other problems associated with IL-2 infusion.

The compositions preferably contain substantially homogeneous populations of cells, such as Th1 cells or Th1-like cells, in which the cytokine profile is predominantly one type of cell (i.e., greater than about 50%). The compositions can contain regulatory immune cells, effector cells or both. In all instances the compositions contain clinically relevant, i.e., a therapeutically effective, numbers of cells.

Such compositions can be used therapeutically to restore an immune cell imbalance. Immune cell imbalances are common in many disease states. For example, a predominance of Th1 regulatory immune cells has been reported in autoimmune diseases such as rheumatoid arthritis [see, Simon, et al. (1994) Proc. Natl. Acad. Sci. U.S.A. 91:8562]; type I diabetes [see, Foulis, et al. (1991) J. Pathol. 165:97]; systemic inflammation [see, Brod, et al. (1991) J. Immunol. 147:810]; inflammatory bowel syndrome [Niessner et al. (1995) Clin. ExD. Immunol. 101:428]; Grave's disease [see, de Carli, et al. (1993) J. Clin. Endocr. Metab. 77:1120]; Sjögren's syndrome [see, Oxholm, et al. (1992) Autoimmunity 12:185]; primary systemic vasculitis [Grau (1990) Eur. Cytokine Netw. 1:203]; and rejected autografts [see, Benvenuto, et al. (1991) Transplantation 51:887]. A predominance of Th2 regulatory immune cells has been reported in AIDS [see, Romagnani, et al. (1994) Res. Immunol. 145:611]; candidiasis [see, Puccetti, et al. (1995) Trends in Microbiology 3:237]; tuberculosis [Zhang, et al. (1995) Infect. Immun. 63:3231]; and allergy [see, Romagnani, et al. (1994) Curr. Opin. Immunol. 6:838].

Also, the polarized Th1 and Th2 responses in humans to different antigens are known to play a role in protection, but also result in immunopathology. The methods provided herein can be used to correct pathologic Th1 and Th2 responses by infusing autologous regulatory cells of the subset in short supply, thereby adjusting the ratios and absolute numbers. Since Th1 and Th2 cells have cross-regulatory properties, large infusions of the subset in short supply can counter-act the pathologic effects of an imbalanced response. Some examples of the use of these methods and cells for treating several disease are provided. It is understood that the following are exemplary uses; any condition in which a pathologic T cell response is observed in which the ratios or amounts of particular subsets of T cells are outside the normal range can be treated by infusion of the T cell subset(s) that is in relatively short supply.

1. Administration

The compositions of cell can be administered by any suitable means, including, but not limited to, intravenously, parenterally, or locally. The particular mode selected will depend upon the particular treatment and trafficking of the cells. Intravenous administration is presently preferred. Typically, about 10¹⁰-10¹¹ cells can be administered in a volume of a 50 ml to 1 liter, preferably about 50 ml to 250 ml., more preferably about 50 ml to 150 ml, and most preferably about 100 ml. The volume will depend upon the disorder treated and the route of adminstration. The cells may be administered in a single dose or in several doses over selected time intervals in order to titrate the dose, particularly when restoration of immune system balance is the goal.

2. Treatment of Autoimmune Disorders

The methods and composition of regulatory cell provided herein may be used to treat disorders that have an underlying autoimmune basis or component.

a. Treatment of Rheumatoid Arthritis (RA)

RA is an immunologically mediated, chronic inflammatory disease characterized by synovial inflammation and autoantibodies. While the underlying cause of RA is unknown, it is well agreed upon that a fault in immune regulation is a principal factor contributing to the disease pathogenesis. Regulated control of normal immune responses are largely the result of interactions between, and the cytokine production of, macrophages, T-cells and B-cells.

Disease activity in RA patients has been positively correlated with the cytokine production of activated macrophages. In an inflamed joint, macrophages produce large amounts of pro-inflammatory cytokines which include IL-1, IL-6, IL-8, TNF-α and GM-CSF. These cytokines act to recruit Th1 memory cells to the joint and stimulate rheumatoid factor (RF) production leading to pannus formation and joint destruction. Treatment protocols which decrease the levels of proinflammatory Th1 cytokines in RA have been shown to result in clinical improvement.

The cytokines IL-4 and IL-10 are known to down-regulate macro-phage activation and inhibit their production of IL-1, IL-6, IL-8 and TNF-α. IL-4 is also capable of suppressing the uncontrolled proliferation of synoviocytes, which is a major pathological feature of RA. IL-4 and IL-10 are produced by Th2 cells, which are virtually absent from the RA joint. Rather, RA joints have an abundance of Th1 cells.

Accordingly, RA can be treated by generating large numbers of autologous, ex vivo derived Th2 cells from RA patients by the methods provided herein. The resulting cells, preferably in amounts greater than 10⁹, more preferably 10¹⁰, are re-infused into the patient to thereby suppress the chronic inflammatory lesions. Th2 cells of memory phenotype are preferred, since memory cells are most likely to migrate to the site of inflammation. In addition, the cells can be infused in an activated state; infiltrating T-cells in RA have been shown to have 5-6 fold increases in HLA-DR expression and 2-5 fold increases in VLA-1 expression, both of which are activation markers.

It is also preferred that the infused Th2 cells only exert their regulatory action in the joints, so as to prevent a systemic immunosuppressive effect. Since the eliciting antigen is unknown in RA, the Th2 cells used should be specific for unique joint antigens [e.g., Type II collagen or proteoglycan].

b. Treatment of Multiple Sclerosis (MS)

MS is an autoimmune disease characterized by central nervous system inflammation and demyelination. The regulation of cytokine spectrum and production in MS is thought to have a decisive influence on disease outcome. Collective data has shown that Th1-associated cytokines, such as TNF-α, lymphotoxin, interleukin-12 and interferon-γ promote disease, while cytokines from Th2 cells, such as IL-10, limit disease. In addition, TGF-β has been shown to be a disease downregulator. Studies in animal models of MS [experimental autoimmune encephalomyelitis (EAE)] have determined that a regulatory cell producing IL-10 and TGF-β, termed “Th3”, has the greatest effect suppressing the development and inducing recovery from disease.

Accordingly, the methods herein can be used to generate therapeutic quantities of Th3 cells from MS patients for use in autologous cell therapy. Since recovery from disease is associated with infiltrating cells which produce IL-10 and TGF-β the ex vivo derived Th3 cells should preferably have a memory phenotype in order to enhance migration to the inflammatory lesions. In addition, in order to make the immunosuppressive effect of the cells specific for the inflammatory lesions, cells specific for myelin or encephalitogenic epitopes of myelin antigens (e.g., myelin basic protein or proteolipid protein) should be used.

C. Inflammatory Bowel Disease (IBD)

IBD is a chronic inflammatory condition of the gastrointestinal tract. The etiology and pathogenesis of IBD is not known. Crohn's disease (CD) and ulcerative colitis (UC) are thought to be mediated by an abnormal or uncontrolled T-cell reaction to one or more common gut constituents. Active CD and UC are characterized by increases in Th1-like cytokines, with little to no detectable Th2-like cytokines.

Accordingly, the methods provided herein can be used to generate autologous Th2 cells for infusion in IDB patients. Preferably, the infused cells will express the integrin, α4, β7. This integrin has been shown to be the ligand for mucosal addressin cell adhesion molecule-1 found on Peyer's patch high endothelial venules, which occur in the gastrointestinal tract. Lymphocytes which express a4, β7 will traffic to and are retained in mucosal organs. The gut mucosa is the site of chronic inflammation in IBD.

d. Treatment of Insulin-Dependent Diabetes Mellitus (IDDM)

IDDM results from the autoimmune destruction of pancreatic islet β cells by the host immune system. The destruction of islet cells is known to be mediated by T-cells. The NOD mouse is a spontaneous model of human IDDM. Islet transplantation as an isograft in these mice can produce normoglycemia and prevent and reverse early complications of diabetes. Host inflammatory responses, however, eventually lead to destruction of the islet transplants and disease recurrence. Analysis of these inflammatory responses has shown that graft specific Th1 cells mediate rejection, while Th2 cells are protective.

There is evidence that isograft and allograft rejection is mediated by Th1 cells and can be suppressed by Th2 cells. Th1 cells have been shown to actively promote diabetes in NOD mice. Inhibition of Th1 cytokines leads to protection of islet isografts in NOD mice. Recently, it has been shown that the systemic administration of Th2 cytokines (IL-4 and IL-10) and adoptive transfer of an islet-specific Th3 clone can inhibit syngeneic islet graft rejection in these animals. Furthermore, Th2-like responses have been shown to be protective in models of allogeneic organ and tissue transplantation.

Accordingly, the methods herein can be used to generate clinically relevant numbers of Th2 cells for infusion in IDDM patients that will protect against rejection of transplanted allogeneic islet cells. Preferably, the Th2 cells will be specific for the allogeneic antigens on the transplanted islets. Alternatively, Th2 cells specific for insulin can be used. Insulin-specific Th2 cells could also be used to treat early diagnosed IDDM patients to prevent islet destruction, as well as used in high risk patients as a vaccine to prevent or at least retard development of the diabetes.

e. Treatment of Other Autoimmune Diseases

Th1-mediated autoimmune diseases, such as, but not limited to, autoimmune thyroid diseases, anti-tubular basement membrane disease (kidney) Sjögren's syndrome, ankylosing spohdylitis, ureoretinitis and others, can be treated by administration of compositions containing a clinically relevant, typically 10⁹-10¹¹, Th2 cells or a Th2-like composition.

3. Transplantation

Th2 cell ACT can be used as an immunosuppressive strategy permitting organ and tissue transplantation. For example, Th2 cytokines have been correlated with non-rejecting heart allografts, while Th1 cytokines correlate with rejection. The same is has been observed for renal allografts and mouse orthotopic liver allografts and skin allografts. Adoptively transferred Th2 cells suppress skin allograft rejection and also allow allogeneic engraftment of spleen cells in sublethally irradiated mice as well as suppress lethal GVHD (graft vs. host disease). T-cell mediated alloreactivity has been shown to be central in the pathogenesis of GVHD and graft rejection.

Accordingly, the methods provided herein can be used to generate autologous Th2 cells for infusion in patients scheduled for organ or tissue transplant. Preferably, the Th2 cells will be specific for the alloantigens or an antigen unique to the organ or tissue being transplanted.

4. Allergic Disorders

Th2 cells appear to have a crucial role in initiating eosinophil infiltration which causes eczematous reactions in patients with atopic dermatitis, and airway hyper-responsiveness and pulmonary eosinophilia in allergic asthma. Furthermore, atopic patients (patients with hayfever, dust and food allergies) have a preferential activation of Th2 cells. Recent evidence has shown that treatments that suppress Th2 development in vivo have profound inhibitory effects on allergen-induced airway changes and other atopic responses. Accordingly, since Th1 cytokines are known to inhibit Th2 responses, the methods herein can be used to generate large numbers of autologous Th1 cells for infusion into atopic patients. Preferably, these cells will be specific for the allergen.

5. Infectious Diseases and Cancer

An excess of Th2 cells is correlated with most infectious diseases, including viral, fungal, yeast, parasitic and mycobacterial infection. In order to change the regulatory balance in favor of cell-mediated immunity, Th1 cells could be infused into these patients. Prior art ACT protocols have used TIL and LAK effector cells and methods that use pathogen- or tumor cell-specific CTLs. These effector cells would not be expected to work properly in an immunocompromised host.

The co-infusion of Th1 regulatory cells should provide the “help” necessary for the effector cells to perform their function and thus improve these therapies. Infusion of Th1 cells alone could provide sufficient help in vivo to drive endogenous CD8+ effector cells.

Accordingly, the methods herein could be used to generate large numbers of autologous Th1 cells for infusion into patients with infectious diseases or cancers. Preferably, the cells will be specific for antigens unique to the pathogen or tumor. The Th1 cells can also be infused with pathogen or tumor-specific cytolytic cells.

Of particular interest herein, are methods for treatment of HIV infection. Methods for producing virally purged CD4+ cells are provided. In preferred embodiments, the cells are expanded under conditions in which Th1 cell differentiation is promoted. The resulting cells are reinfused into the donor HIV patient, whereby immunity will be restored. In other embodiments, these cells are reinfused with expanded effector cells, particularly effector cells that are specifically targeted against HIV infected cells.

Other infectious diseases that can be treated with Th1 cell compositions include, but are not limited to: influenza viruses, polio virus, leukemia viruses, hepatitis viruses, respiratory synctial virus, herpes viruses, retroviruses Epstein-Barr virus, syphillis (Treponema pallidum), cutaneous T-cell lymphoma (mycosis fungoides), Rhodococcus equi (intracellular respiratory pathogen), hypersensitivity pneumonitis, onchocercal keratitis (river blindness), burn victims, chlamydia trachomatis, mycobacterium avium, candida albicans, coxackievirus, Leishmania major infection, cryptococcal infection and Bordetella pertussis respiratory infection.

Infectious diseases that can be treated with Th2 cell compositions include, but are not limited to: filarial nematode (parasite), Plasmodium chaboudi chaboudi (malaria), and Borrelia burgdofi (spriochete) infections.

Also of interest herein, are methods of treatment of cancer. In preferred embodiments, methods for treatment of renal cell carcinoma are provided. Transformed renal cells express heat shock protein hsp70. Consequently, hsp70-specific Th1 cells could serve as a cytokine delivery vehicle to increase local concentrations of IL-2 and IFNγ in the tumor, thereby promoting anti-tumor effector cell function, activity and/or proliferation.

Th1 cells can also be used to mediate tumor regression in cancers including melanoma, breast cancer, head and neck cancer, prostate cancer and lung cancer. These is evidence that for certain tumors, a Th2 rsponse may mediate regression.

6. Vaccination

The development of effective vaccine strategies for intracellular pathogens, including, but not limited to, bacteria, viruses and parasites, is one of the major frontiers of medical research. Research centers on antigens from pathogenic organisms and adjuvants that can elicit a Th1-like response in patients. It is known that a Th1 response is protective for infectious pathogens. Th1 responses are weak or non-existent in some patients with most vaccine protocols. Other research focuses on eliciting an IgA antibody response, which is thought to be protective against organisms that enter the body through muscous membranes. An IgA response is mediated by Th2 cells. To better control the type of immune response a patient will elicit to a vaccine, the methods herein provide a means for ex vivo vaccination (i.e., the addition of the vaccine antigen(s) to patient mononuclear cells ex vivo, whereby the cells are activated under conditions that promote the desired regulatory cell differentiation.

The methods provided herein can be used to withdraw blood from a patient, expose the isolated mononuclear cells to the vaccine antigen in the presence of IL-12 and/or IFN-γ and/or IL-4, and expand the Th1 or Th2 cells for reinfusion. Preferably, the cells used will have a memory phenotype so they will provide long-term protection. CD4+ and CD8+ Th1 or Th2 cells could be generated alone or in combination.

The following examples are included for illustrative purposes only and are not intended to limit the scope of the invention.

### EXAMPLE 1

Screening Mitogenic Monoclonal Antibodies

This example demonstrates a method for identifying antibodies that are suitable for expanding T-cell subsets, either singly or in combinations thereof. In order to determine co-stimulatory signals required for T-cell subset proliferation, cells are incubated with various monoclonal antibodies (mAb) and their proliferation determined in ³H-thymidine incorporation assays. To exemplify this procedure, the following experiments were conducted.

Monoclonal Ab to CD3 (64.1, IgG2a) and anti-CD5 (10.2, IgG2a) were gifts from J. Ledbetter (Bristol Meyers, Seattle) and the mAb to CD28 (Kolt-2, IgG1) was a gift from K. Sagawa (Kurume University, Kyushu, Japan). These mAb were purified from ascites fluids on protein A sepharose columns. All other mAbs were purchased from PharMingen (San Diego, Calif.). All mAbs were dialyzed against phosphate buffered saline and filtered through sterile 0.45 μm filters.

Goat anti-mouse affinity purified antibody (Tago, Burlingame, Calif.) was immobilized on plastic 96 well tissue culture plates. The antibody was dissolved in sodium borate buffer (pH 8.6) at a concentration of 10 μg/ml and 100 μl was placed in each well. Plates were washed three times with RPMI-1640 with 10% normal human serum. Cells were labelled with anti-CD3 mAb (1 μg/ml) on ice for 15 minutes prior to plating. 50,000 cells were plated in each well. Co-stimulatory mAbs were added in the soluble phase at 1 μg/mi. The cells were cultured at 37° C. in an atmosphere of 5% CO₂. After 88 hours of culture, cells were pulsed with 1 μCi of [³H]- thymidine (specific activity of 2 Ci/mole, New England Nuclear). Eight hours later, cells were harvested with a PHD cell harvester (Cambridge Technology, Cambridge, Mass.) and the radioactivity on the filter papers counted on a liquid scintillation counter (LS1701, Beckman).

The results of mAb addition to purified CD4+ and CD8+ cells from a normal individual are shown below. Results are shown as mean counts per minute (cpm) of four replicates. Standard errors were always less than 10%.

| Stimulation | CD4+ | CD8+ |
|---|---|---|
| medium alone | 320 | 484 |
| anti-CD3 | 582 | 541 |
| anti-CD3+ anti- | 18,450 | 17,222 |
| CD5 | | |
| anti-CD3+ anti- | 20,400 | 18,641 |
| CD28 | | |
| anti-CD5 | 450 | 246 |
| anti-CD28 | 826 | 821 |

These data demonstrate that anti-CD5 and CD28 are capable of providing a co-stimulatory signal for T-cell proliferation in CD4+ and CD8+ subsets when the cells are activated with anti-CD3. The results of combining anti-CD5 and CD28 are shown below:

| Stimulation | CD4+ | CD8+ |
|---|---|---|
| medium | 428 | 524 |
| anti-CD3 | 585 | 508 |
| anti-CD3+ anti-CD5 | 13,422 | 10,080 |
| anti-CD3+ anti-CD28 | 14,628 | 12,821 |
| anti-CD3+ anti-CD5+ anti-CD28 | 25,248 | 29,804 |
| anti-CD3+ IL-2 (10 U/ml) | 11,428 | 12,401 |

These results show that the combination of anti-CD5 and anti-CD28 as co-stimulatory signals in CD3 activated, purified T-cells induces a greater proliferative response than either mAb alone. In addition, the combined mAbs generated a proliferative response without addition of IL-2.

The effect of various mAbs (second signal) on purified CD8+ cells from a normal donor used in conjunction with anti-CD3 or anti-CD2 (first signal) was also tested. These results are shown below:

| Stimulation | αCD3 | αCD2 | Medium |
|---|---|---|---|
| αCD5 | 206 | 193 | 155 |
| αCD8 | 787 | 578 | 640 |
| αCD11a | 949 | 830 | 840 |
| αCD27 | 844 | 2 | 788 |
| αCD28 | 1928 | 529 | 640 |
| αCD44 | 779 | 477 | 498 |
| aCD45RO | 3199 | 1878 | 1978 |
| IL-2 | 4347 | 1834 | nd |
| Medium | 289 | 217 | 212 |

These results demonstrate that anti-CD3 as the first signal delivers a more powerful proliferative stimulus than anti-CD2. Anti-CD45RO and anti-CD28 mAbs appear to deliver the strongest second or co-stimulatory signals when used with anti-CD3.

Combinations of these antibodies were tested on anti-CD3 activated, ex vivo generated CD8+ cytolytic cells specific for the MAGE-3 antigen on melanoma cells. These results are shown below:

| | anti-CD11a | anti-CD27 | anti-CD28 | anti-CD45RO |
|---|---|---|---|---|
| anti-CD11a | — | 1365 | 1116 | 1208 |
| anti-CD27 | 1365 | — | 374 | 973 |
| anti-CD28 | 1116 | 374 | — | 948 |
| anti-CD45RO | 665 | 973 | 948 | — |

Combinations including anti-CD11a provided the strongest proliferative signals for these cells. None of these combinations provided very exceptional growth. This sometimes occurs in CD8+ CTL, which are unable to produce sufficient endogenous cytokines. Co-culturing of these cells with autologous CD4+, however, enhanced the proliferation of these cells with mAb stimulation. This probably resulted from the increased endogenous production of IL-2, as well as IFN-γ and IL-7.

### EXAMPLE 2

CD4+ and CD8+ T-cells from Normal Donor

This example demonstrates that polyclonally activated CD4⁺ and CD8⁺ regulatory T-cell subsets can be expanded without IL-2 to clinically relevant numbers from a starting number of about 1×10⁶ cells using the disclosed methods.

A. Collecting Mononuclear Cells

Mononuclear cells from normal donors were obtained from source leukocyte packs (Interstate Blood Bank, Inc.). The leukopack cells were diluted 1:1 with Hank's Buffered Salt Solution (HBSS) without calcium (Ca²⁺) or magnesium (Mg²⁺) and 30 to 35 ml of the diluted cells were placed over 12 ml of Ficoll-Hypaque and the tube centrifuged at 1500 RPM at room temperature. The buffy coat layer containing lymphocytes and monocytes was transferred by Pasteur pipette to a clean 50 ml centrifuge tube and washed three times with HBSS. The cells were then resuspended in RPMI-1640 medium supplemented with 10% human serum, 25 mM HEPES buffer, 2.0 mM glutamine, 1.0 mM sodium pyruvate, 0.1 mM non-essential amino Acids, 2×10⁻⁵ M 2-mercaptoethanol, 10 IU of penicillin G and 100 mg/ml streptomycin sulfate (cRPMI). The monocytes were depleted by adherence to plastic T-cell flasks incubated overnight at 37° C. in an atmosphere of 5% CO₂ and 100% humidity.

B. Precursor Cell Purification

T-cell subsets were purified with immunomagnetic bead technology. GAM-coated beads (Dynal, Inc.) were washed twice with HBSS and incubated overnight on a rotating wheel at 4° C. in HBSS with 1% normal human serum in order to block nonspecific binding. The non-adherent cells were incubated with either anti-CD4 or anti-CD8 mAb at pre-titered concentrations on ice for 30 minutes. Labelled cells were washed twice and resuspended in cRPMI at 10 cells/ml. The beads were added to the cells at a bead/cell ratio of 2:1 and mixed well. This mixture was gently centrifuged at 500 RPM for 1 minute at 4° C. The bead/cell mixture was then resuspended by gently inverting the centrifuge tube. The tube was then placed on a rotating wheel for 30 minutes at 4° C. The bead/cell mixture was then diluted 5 fold with cRPMI and placed on a cobalt salarium magnet. The supernatant was aspirated and rosetted and the procedure repeated. The rosettes were incubated for 24 hours in cRPMI at 37° C. in an atmosphere of 5% CO₂. After 24 hours, the majority of cells detached from the beads and the beads were removed by placing the solution back on the magnet. The resulting cells were greater than 98% pure CD4⁺ or CD8⁺ T-cells as assessed by flow cytometry.

C. Ex Vivo Differentiation

The purified CD4+ cells were divided into twoeparate groups of 1 million cells each. The first group was activated with immobilized anti-CD3 mAb in the presence of 400 U/ml of IL-4 and 10 μg/ml of anti-IFN-γ mAb and anti-CD28 mAb. This first group (Th2) was expanded under these conditions for another 10 days. The second group was activated with immobilized anti-CD3 in the presence of 25 U/ml of IL-12 and 150 U/ml of IFN-γ, and anti-CD28 mAb. These cells were harvested and washed after 6 days of culture.

D. Regulatory Cell Expansion

One million of each of the purified T-cell subsets were labelled for 30 minutes on ice with anti-CD3 mAb (64.1, lgG2a). 2.5×10⁵ cells of the purified CD4⁺ and CD8⁺ cells were suspended in 1 ml of cRPMI and plated into 4 separate wells of a 24-well plate coated with goat anti-mouse (GAM) polyclonal antibody. Purified anti-CD5 (10.2, IgG2a) and anti-CD28 (KOLT-2, IgG1) mAb were added to the wells at a final concentrations of 200 ng/ml. The cells were then incubated at 37° C. in an atmosphere of 5% CO₂.

After 3 days, 1 ml of cRPMI with 200 ng/ml of anti-CD5 and anti-CD28 was added to the wells. After 6 days, the wells were harvested, pooled and washed twice in cRPMI. The viable cells were counted and resuspended in cRPMI at 1×10⁶ cells/ml and incubated in T-flasks for 48 hours at 37° C. The cells were then harvested, washed twice, labelled with anti-CD3 mAb on ice for 30 minutes and inoculated into the extra capillary space of a GAM-coated mini-hollow fiber bioreactor with 200 ng/ml of anti-CD28 an danti-CD5 mAb. The cells were harvested, washed and counted after 14 days.

1. Mini-Hollow Fiber Bioreactor

A mini-hollow fiber device was constructed to expand immune effector cells. The device had four mini-hollow fiber units in parallel. The hollow fibers (CD Medical, Hialeah, Fla.) had a 9 ml extracapillary volume and the fibers had molecular weight cut offs of 10,000 daltons. The hollow fibers were coated with GAM polyclonal antibody. Coating was accomplished by dissolving GAM polyclonal antibody, at a concentration of 10 mg/ml, in sodium borate buffer (pH 8.6) and inoculating the sterile solution into the extracapillary space (ECS) of the hollow fiber bioreactors. The lumenal and ECS ports were then sealed and the bioreactors placed on a rotating plate and incubated at 4° C. for 24 hours. Prior to use, the bioreactors were washed with phosphate buffered saline with 1% normal human serum.

The flow path included an integration vessel, pump and oxygenation cartridge. Luminal flow rates ranged between 100 and 400 ml/minute and were increased manually proportionate with the cell growth in the bioreactors. The pH and temperature were continually monitored and controlled by microprocessor. The pH was adjusted and maintained at 7.2 by altering the speed of fresh medium fed into the integration vessel and the percent CO₂ in the oxygenation cartridge. The temperature was controlled to 37° C. by adjusting the wattage to a heating coil wrapped around the integration vessel.

2. Single Large Hollow Fiber Bioreactor

The cells recovered from the mini hollow fiber device were incubated in T-flasks at 1×10⁷ cells/ml in cRPMI without mAb stimulation for 48 hours. The cells were then labelled with anti-CD3 mAb and inoculated into a GAM-coated large hollow fiber bioreactor [see, copending allowed U.S. application Ser. No. 08/506,173, discussed above] with 200 ng/ml of anti-CD5 and anti-CD28 mAb. The cells were harvested, washed and counted after 14 days.

3. 8-Cartridge Hollow Fiber Bioreactor

The cells recovered from the single large hollow fiber bioreactor [see, copending allowed U.S. application Ser. No. 08/506,173, discussed above] were incubated for 48 hours in a 10 liter spinner flask at 10⁷ cells/ml in cRPMI without mAb stimulation. The cells were then labelled with anti-CD3 mAb and inoculated into each of the 8 GAM-coated hollow fiber bioreactors with 200 ng/ml of anti-CD5 and anti-CD28 mAb. After 14 days, the cells were harvested, washed and counted.

E. Results

Clinically relevant numbers of cells were produced as follows:

| Day | CD4⁺ (Th1) | CD4⁺ (Th2) | CD8⁺ | Culture Vessel |
|---|---|---|---|---|
| 0 | 1 × 10⁶ cells | 1 × 10⁶ cells | 1 × 10⁶ cells | 24-well plate |
| 6 | 1.3 × 10⁷ cells | 7.2 × 10⁶ cells | 9.8 × 10⁶ cells | 24-well plate |
| 8 | 1.0 × 10⁷ cells | 6.5 × 10⁶ cells | 6 × 10⁶ cells | Mini-HF |
| 22 | 1.3 × 10⁹ cells | 1.0 × 10⁹ cells | 1.2 × 10⁹ cells | Mini-HF |
| 24 | 1.1 × 10⁹ cells | 1.0 × 10⁹ cells | 1.1 × 10⁹ cells | 1-large HF |
| 38 | 1.4 × 10¹⁰ cells | 1.0 × 10¹⁰ cells | 1.2 × 10¹⁰ cells | 1-large HF |
| 40 | 1.3 × 10¹⁰ cells | 1.0 × 10¹⁰ cells | 1.0 × 10¹⁰ cells | 8-Large HF |
| 54 | 1.1 × 10¹¹ cells | 1.0 × 10¹¹ cells | 9.9 × 10¹⁰ cells | 8-Large HF |

Therefore, compositions containing clinically relevant numbers of T-cell subsets can be produced.

### EXAMPLE 3

Virus-purged CD4⁺ Th1-cells from HIV⁺ Patient

This example demonstrates that clinically-relevant numbers of virus-purged CD4⁺ Th1-cells can be generated by the methods herein for use as an ACT for A.I.D.S. The cells were purged of active virus by selection of CD4 antigen and were polyclonally activated and again selected for CD4 antigen to purge of latent virus.

A. Obtaining Mononuclear Cells

An HIV⁺ patient, identified by a routine blood screening procedure confirmed by Western Blot analysis, in WHO stage IV was the donor for this study. The patient underwent a leukopheresis procedure for collection of peripheral blood mononuclear cells.

B. Regulatory Cell Purification

CD4⁺ cells were isolated by positive selection on immunomagnetic beads as described above. The CD4⁺ cells were then activated in 24-well plates with immobilized anti-CD3 mAb and in the presence of 40 U/ml of interferon-γ (IFN-γ). After 24 hours in culture, the cells were harvested, washed and re-selected for CD4 on immunomagnetic beads. The positively-selected cells were labelled with anti-CD3 mAb and plated at 25,000 cells/well in a GAM-coated 96-well plate in cRPMI. Anti-CD28 mAb and IFN-γ was added to the wells at a concentration of 1 μg/ml and 40 U/ml, respectively. After 7 days, supernatant from each well was tested for p24 antigen with a commercial ELISA assay (Dupont). All negative wells were pooled, relabelled with anti-CD3 mAb and re-plated at 25,000 cells/well in a GAM-coated 96-well plate in cRPMI with anti-CD28 mAb.

C. Regulatory Cell Expansion

The cells were expanded as described in Example 2 above, except that only anti-CD28 mAb was used as a co-stimulatory agent.

D. Results

6.3×10¹⁰ cells were grown over a 72 day period. The cells were negative for p24 antigen and were capable of producing IL-2 and IFN-γ, but little or no IL-4. The cells were also shown to be capable of providing help for NK-function in a dose-dependent manner. The cells were reinfused into the patient. Reinfusion of these cells into the HIV⁺ patient should be a treatment for A.I.D.S.

### EXAMPLE 4

HIV-specific CD8⁺ Cells from a HIV⁺ Donor

This example demonstrates that antigen-specific CTL can be purified and expanded from an individual with a viral infection.

A. Obtaining Effector Cells

3×10⁸ mononuclear cells were obtained by leukaphoresis from a stage IV A.I.D.S. patient. CD8⁺, CD25⁺ cells were purified by two rounds of selection on immunomagnetic beads.

B. Expansion of Effector Cells

Approximately 2×10⁶ cells were recovered and expanded in a 24-well plate coated with anti-CD3 mAb and with soluble anti-CD28 mAb. After 6 days, the cells were washed (×2) and inoculated into mini-hollow fiber bioreactors. After 18 days in the mini-hollow fiber units, the cells were washed, counted and allowed to rest 2 days before inoculation into a cartridge of the large hollow fiber bioreactor under the same conditions as described in Example 2 above.

After 16 days, the cells were harvested, washed and allowed to rest for 2 days. The viable cells were then inoculated into the 8-cartridge hollow fiber bioreactor system and cultured under the same conditions as described in example 2 above.

C. Results

6×10¹⁰ viable cells were harvested after 20 days. The cells showed significant Ag-specific CTL activity against infected autologous cells.

These cells can be reinfused into the patient as a treatment for A.I.D.S. In addition, these can be co-infused with virally-purged CD4⁺, produced as described in EXAMPLE 3.

### EXAMPLE 5

Antigen-specific Th2-like Cells from a Normal Donor

This example demonstrates that antigen-specific Th2-like CD4⁺ cells can be derived from a normal individual and expanded to clinically relevant numbers.

A. Obtaining Regulatory Cells

50 ml of blood was collected into a heparinized syringe, using sterile technique, from an HIV⁻ volunteer. Peripheral blood mononuclear cells (PBMC) were separated by Ficoll-Hypaque density gradient centrifugation. The PBMC were cultured in 10 ml T-flasks at 2×10⁶ cells/ml and pulsed with gp 120 antigen in cRPMI that contained 1.0 μg/ml of anti-IFN-γ mAb and 20 U/ml of IL-4. After 2 days, the blasts were collected by selection of CD25 on immunomagnetic beads. The blasts were allowed to rest for 72 hours and were than re-stimulated with gp-120 pulsed, autologous monocytes and immediately cloned in soft agar. The small number of cells that survived and grew out as colonies (1/150,000) were enriched in Ag-specific cells that produced IL-4 and IL-10 and little IFN-γ upon stimulation, and, thus, were Th2-like in cytokine profile.

B. Expansion of Effector Cells

The cells were expanded as described in Example 2 and grew to 9×10¹⁰ cells in 62 days.

### EXAMPLE 6

Differentiation of Th2 Cells from Precursors in Rheumatoid Arthritis Peripheral Blood

While T cell cytokine expression is very low in rheumatoid arthritis (RA), the absence of Th2 factors (e.g., IL-4 and IL-13) is especially striking. Since Th2 cytokines suppress production of pro-inflammatory cytokines, metalloproteinases and rheumatoid factor, their relative absence in RA could contribute to disease perpetuation. The lack of Th2 cells in synovium suggests that this differentiation pathway might be defective in RA. To determine if Th2 precursors are present in RA, the ability of peripheral blood RA CD4+ T cells to differentiate into Th0 (IL-4 +IFN-λ), Th1 (IFN-λ, no IL-4) and Th2 cells (IL-4, no IFN-λ) in vitro was studied.

Purified CD4+ T cells were cultured in the presence of immobilized αCD3 antibody, αIL-12 and IL-4 for 3 d. Cells were then washed and stimulated with PMA and ionomycin in the presence of monensin for 6 hr. The cytokine phenotype was determined using 2-color flow cytometry on permeabilized cells with αIL-4 and βIFN-λ monoclonal antibodies. The results are shown as percent cells±standard error (se); “n” values are in parentheses.

| | Treatment | Th2(%) | Th0(%) | Th1(%) |
|---|---|---|---|---|
| RA (9) | αCD3 | 0.68 ± 0.19 | 0.44 ± 0.11 | 10.38 ± 2.61 |
| Normal (6) | | 0.56 ± 0.08 | 0.55 ± 0.17 | 11.07 ± 2.89 |
| RA (4) | αCD2+ IL-4 | 1.43 ± 0.32* | 0.29 ± 0.09 | 4.68 ± 0.91 |
| Normal (5) | | 1.50 ± 0.26* | 1.69 ± 0.56 | 13.27 ± 2.46 |
| RA (6) | αCD3+ | 3.03 ± 0.92* | 1.68 ± 0.44 | 12.51 ± 3.15 |
| Normal (3) | αIL-12+ IL-4 | 1.45 ± 0.35* | 0.72 ± 0.36 | 7.30 ± 0.84 |

These data indicate that similar numbers of Th2 cell precursors are present in the peripheral blood of normals and patients with RA. Furthermore, the mature Th2 cell population can be significantly increased (p<0.05) with IL-4 and α-IL-12 antibody. Hence, a specific Th2 precursor defect does not account for the cytokine profile in the joint. This raises the possibility that novel therapeutics could be developed involving the administration of ex vivo differentiated and expanded Th2 cells.

### EXAMPLE 7

HIV+ Lymphocyte Proliferation

The ability of PBL from HIV+ donors to proliferate in response to the polyclonal activator PHA-P and immobilized anti-CD3 mAb was compared with PBL from a normal donor (Table 1). PBL from HIV+ donors exhibited a marked suppression in the ability to respond to either mitogenic signals when compared to PBL from normal donors.

TABLE 1
Comparison of Proliferative Response of Normal
and HIV+ PBL to Mitogenic Factors*

| | | | Immobilized |
|---|---|---|---|
| PBL Source | Medium Alone | PHA-P (1 ng/ml) | anti-CD3 mAb |
| normal donors | 1,446 ± 241 | 25,813 ± 1200 | 27,206 ± 1891 |
| HIV+ donors | 2,041 ± 421 | 5,680 ± 460 | 4,204 ± 562 |

*Peripheral blood lymphocytes (PBL) isolated over Ficoll-Hypaque were plated at 50,000 cells/well in 96-well flat bottom culture plates. Cells were pulsed after 88 hours of stimulation with medium alone, PHA-P or immobilized anti-CD3 mAb with [³H]-thymidine for eight hours and the average mean and standard error of quadruplicate samples for six normal and six HIV+ individuals is shown in cpm.

To determine if purified T-cell subsets from HIV+ donors were capable of responding to mitogenic stimuli in the absence of activator, the following study was conducted. PBL from six normal and six HIV+ individuals (same individuals as used in the experiments shown in Table 1) were incubated in plastic tissue culture dishes for 24 hours at 37° C. in an atmosphere of five percent CO₂ in air. The CD4+ and CD8+ T-cell subsets were purified using positive selection on immunomagnetic beads as described previously. The results are shown in Table 2.

TABLE 2
Proliferative Response of Normal and HIV+ T-Cell
Subsets to Mitogens

| | | Immobilized anti- | |
|---|---|---|---|
| | Medium | CD3⁺IL-2 | PMA |
| (purity %) CD4⁺ | | | |
| (99.5) Normal donors | 1,841 ± 320 | 42,186 ± 3444 | 35,920 ± 3420 |
| (98.8) HIV+ donors | 1,346 ± 230 | 29,212 ± 1841 | 31,440 ± 6210 |
| (purity %) CD8⁺ | | | |
| (98.8) Normal donors | 1,925 ± 421 | 12,420 ± 821 | 10,920 ± 1104 |
| (98.4) HIV+ donors | 1,212 ± 168 | 10,861 ± 948 | 6,155 ± 718 |
| *T-cell subsets isolated by positive selection on immunomagnetic beads from six normal and six HIV+ donors. Average purities are shown in parenthesis. The cells were plated at 50,000 cells per well in 96 well flat bottom tissue culture plates in CRPMI and 10 percent NHS pulsed for eight hours with 1 μCi [³H]-thymidine after 88 hours of stimulation with either medium alone, immobilized anti-CD3+ IL-2 (10 u/ml) or PMA (0.5 ng/ml). | | | |
| # Results are shown as the average cpm and standard errors, Each group was performed in triplicate. | | | |

The results indicate that a significant T-cell proliferative response is possible from HIV+ donors. The CD4+ cell response to anti-CD3+ IL-2 of HIV+ donor cells was approximately 30 percent less than for the normal donors, but still significantly higher than the medium alone control. The CD8+ cells of HIV+ donors responded nearly the same to anti-CD3+ IL-2 as did normal cells. The CD8+ response of normal and HIV+ donor cells was significantly less than that observed in CD4+ cells. These results indicate that purified T-cell subsets from HIV+ donors are capable of responding to mitogenic signals.

To demonstrate that mitogenic mAbs could provide the second signal for T-cell proliferation in anti-CD3 activated T-cells from HIV+ donors the following experiments were performed. T-cells purified from PBL of HIV+ donors were isolated using AET-treated SRBC. The anti-CD3 activated T-cells were exposed to soluble anti-CD8 alone, anti-CD5 alone and a combination of anti-CD28 and anti-CD5. The results are shown in Table 3.

TABLE 3
Proliferation Response of T-Cells from HIV+ Donors
to Mitogenic mAbs*

| Stimulation | cpm ± SEM |
|---|---|
| medium | 1,810 ± 130 |
| anti-CD3 | 2,338 ± 144 |
| anti-CD3± IL-2 | 11,882 ± 35 |
| anti-CD3± anti-CD28 | 13,334 ± 300 |
| anti-CD3± anti-CD5 | 3,629 ± 102 |
| anti-CD3± anti-CD5+ anti-CD28 | 12,882 ± 69 |
| *T-cells purified by the AET-treated SRBC E-rosetting procedure (99.6 percent CD3+) were isolated from PBL of an HIV+ donor. The cells were plated at 50,000 per well in a 96 well flat bottom tissue culture plate in cRPMI and 10 percent NHS. The cells were activated with immobilized anti-CD3 mAb and stimulated with either IL-2 (10 u/ml), soluble anti-CD28 mAb (200 ng/ml) soluble anti-CD5 (200 ng/ml) or a combination of soluble anti-CD8 and anti-CD5. Cells were pulsed for eight hours | |
| # with 1 uCi [³H]-thymidine after 88 hours of stimulation. Results are shown as cpm and standard error from a single donor. Each treatment group was run in guadruplicate. | |

Anti-CD28 was as effective as IL-2 in providing the second signal to purified T-cells from an HIV+ donor. Anti-CD5 had no effect alone or in combination with anti-CD28 while augmenting the proliferative response in T-cells from normal donors.

Minimum Cell Density Required for Proliferative Response.

In order to determine the minimum cell density required for the immobilized anti-CD3/soluble anti-CD28 system to cause 7-cells from HIV+ donors to proliferate, the following study was conducted.

T-cells from an HIV+ donor and a normal donor were purified using the AET-treated SRBC E-rosette procedure described earlier. Purities of T-cells were 99.4 percent for the HIV+ donor and 99.2 percent for the normal donor. The T-cells were serially diluted from a starting concentration of 1×10⁶ cells/ml and plated onto 96 well plates. Final cell count/well ranged from 100,000 to 1,000. All experimental groups were studied in quadruplicate. The results are shown in Table 4.

TABLE 4
Minimum Cell Density Required for
T-Cell Proliferative Response in the
Anti-CD3/Anti-CD28 System

| HIV+ Donor | | | Normal Donor | |
|---|---|---|---|---|
| # Cells/ | | Anti-CD3 | | Anti-CD3 |
| Well | Medium | Anti-CD28 | Medium | Anti-CD28 |
| 100,000 | 1,628 ± 42 | 22,842 ± 462 | 1,042 ± 214 | 52,820 ± 428 |
| 50,000 | 1,822 ± 120 | 14,920 ± 108 | 1,944 ± 108 | 29,642 ± 262 |
| 25,000 | 1,206 ± 24 | 8,444 ± 48 | 1,496 ± 51 | 14,322 ± 125 |
| 10,000 | 1,828 ± 18 | 2,420 ± 186 | 1,684 ± 49 | 6,246 ± 68 |
| 5,000 | 1,484 ± 56 | 1,848 ± 342 | 1,544 ± 32 | 4,820 ± 320 |
| 1,000 | 1,741 ± 85 | 1,296 ± 260 | 1,821 ± 74 | 1,948 ± 146 |
| *T-cells purified by an E-rosetting procedure using AET-treated SRBC from a normal and an HIV+ donor were tested for their ability to respond to immobilized anti-CD3 mAb and 200 ng/ml of soluble anti-CD28 mAb. T-cells were cultured for 88 hours with anti-CD3/anti-CD28 or medium alone and then pulsed with [³H]-thymidine for an additional eight hours. Results are shown as cpm± standard error. | | | | |
| # All treatment groups were run in duplicate. A single donor was used in each treatment group. | | | | |

T-cells from the HIV+ donor exhibited significant proliferative response in the anti-CD3/anti-CD28 system at cell densities above 2.5×10⁵ cells/ml (25,000 cells per well). T-cells from the normal donor were capable of responding down to a density of 5×10⁴ cells/ml (5,000 cells/well). The proliferative response of T-cells from the HIV+ donor was approximately 50 percent less than the T-cells from the normal donor.

HIV Purge Method

H9 Continuous Cell Line.

In order to reconstitute the Immune system of an AIDS patient, large numbers of CD4+ cells are required. Since these cells harbor latent and active HIV-1, a method is required that will isolate a viral-free starting population of CD4+ cells. If the purging method is not 100 percent effective, the virus will quickly take over the culture as it is stimulated to replicate by activation of the host cell.

To demonstrate the feasibility of purging CD4+ cells from AIDS patients of HIV-1, an HIV-infected continuous cell line was used. The cell line, H9 (gift from Dr. Gallo, NIH, deposited under ATCC No. CRL 8543), is a cloned CD4+ human lymphocyte line. It grows continuously in culture and can also continuously propagate HIV-1.

p24 ELISA.

A commercial kit (Dupont) was used to assay the amount of virus in the cell cultures and monitor the efficiency of the purging experiments. The kit can detect one viral particle in 5,000 cells. The test uses highly specific rabbit polyclonal antibodies to HIV p24 core antigen. These antibodies are immobilized on a 96-well plate. The antibodies capture p24 antigen that is released into the supernatant of a cell culture after treatment with five percent triton-X to lyse the cells. The captured p24 core antigen is then complexed with anti-p24 biotinylated polyclonal antibodies. The complexes are probed with a streptavidin-HRP (horseradish peroxidase) conjugate. The complexes are detected by incubation with orthophenyidiamine-HCl (ORD) which produces a yellowish color proportional to the amount of HIV p24 antigen captured. The absorbance of each well was determined on a microplate reader (Dynatech, Minireader II) and calibrated against the absorbance of known values of p24 antigen. To increase the sensitivity of the test, test cells were co-cultured with PHA-activated, normal lymphocytes.

Results

The theory used for the purging protocol is based on known phenotypic behavior of infected cells. HIV+ cells with active virus will express the env gene products gp120 and gp4l on their cell surfaces. Since it was reported that HIV+ cells with active virus internalize their CD4 receptors, positive selection of CD4 was tested.

H9 cells not infected with HIV-1 are 85 percent CD4+ (H9−) whereas infected H9 cells (H9+ ) are four percent CD4+ as determined by flow cytometry. An experiment was designed where 10 million H9 cells were mixed in the following ratios:

(1) 10 percent H9+ and 90 percent H9;

(2) 30 percent H9+ and 70 percent H9:

(3) 60 percent H9+ and 30 percent H9; and

(4) 80 percent H9+ and 20 percent H9

Cells from each group were positively selected for CD4 with immunomagnetic beads. A sample of the positively selected cells were tested for p24 with the commercial ELISA test (no co-cultivation). Results are shown in Table 5.

TABLE 5
Purge of H9 Cells Infected with HIV-1.

| | p24 before CD4 removal | p24 after CD4 removal |
|---|---|---|
| 0% H9+ | 0.03 ng | 0.01 ng |
| 10% H9+ | 0.25 ng | 0.00 ng |
| 30% H9+ | 0.58 ng | 0.00 ng |
| 60% H9+ | 0.94 ng | 0.03 ng* |
| 80% H9+ | 1.36 ng | 0.03 ng* |
| 100% H9+ | 2.14 ng | 0.09 ng |

*same as negative control

The continuous cell line H9 infected HIV-1 (H9+) and non-infected H9 (H9−) were mixed at various ratios. Cells expressing the CD4 surface antigen were purged from the mixture using specific mAbs and immunomagnetic beads. The amount of p24 antigen in the cultures was determined before and after the purge process.

All groups with the exception of the 100 percent H9+ group were successfully purged of virus below the detectable limits of this assay. To determine if the negative fractions would continue to be viral-free the cells were incubated for 20 days in 24-well plates with 3×10⁶ indicator cells (normal lymphocytes activated with PHA for 72 hours) In cRPMI and 10⁹ NHS. Fresh indicator cell were added again on day seven. On days seven, 14 and 20, 1×10⁸ cells from each group were lysed with triton-X and assayed for p24. The results are shown in Table 6.

TABLE 6
Co-Cultivation of Viral Purged H9 Cells with Indicator Cells

| Day | 10% H9+ | 30% H9+ | 60% H9+ | 80% H9+ |
|---|---|---|---|---|
| 0 | 0.00 ng | 0.00 ng | 0.03 ng | 0.03 ng |
| 7 | 0.04 ng | 0.14 ng | 0.20 ng | 0.29 ng |
| 14 | 0.09 ng | 0.23 ng | 0.38 ng | 0.32 ng |
| 20 | 0.25 ng | 0.53 ng | 0.59 ng | 0.38 ng |

H9+ cells mixed with H9− cells at various ratios were purged of CD4+ cells using immunomagnetic beads. The H9− fractions were co-cultured with PHA-stimulated lymphocytes. The fractions were tested for presence of p24 viral antigen at days zero, seven, 14 and 20.

These results indicate that the original viral purge was not 100 percent effective and virus can still exist below the level of sensitivity of the assay. In a further attempt to develop a viral-free culture, 1×10⁶ cells from each group were serially diluted and plated at 500 cells per well in 2,000 wells of 24-well plates. The cells were allowed to expand for 14 days and then were co-cultured with indicator cells for 20 days as before. Cell samples were analyzed for p24 antigen after 20 days as described earlier. The results are shown in Table 7.

TABLE 7
Co-Culture of Viral-Purged H9 Cells with indicator Cells
After Plating at 500 Cells/Well

| Group | % of Positive Wells* |
|---|---|
| 10% H9+ | 16% |
| 30% H9+ | 32% |
| 60% H9+ | 26% |
| 80% H9+ | 32.5% |

*any value over the negative control
H9+ cells mixed with H9− cells at various ratios, purged of CD4+ cells and cultured for 20 days with PHA-stimulated indicator lymphocytes were serially diluted to 500 cells per well of a 24-well plate. The cells were allowed to expand for 14 days and assayed for p24 viral antigen. The percent of wells from each ratio of H9+ to H9− cells that were positive for p24 is shown.

Those results showed that virally-infected cells could be eliminated after positive selection by serial dilution. To further validate this procedure, the negative wells were pooled and cultured with indicator cells for another 20 days. All groups remained negative for p24 antigen (data not shown). Thus, the combination of positively selecting CD4+ cells followed by serial dilution, should be useful as a viral purge method.

To further test the sensitivity of the assay system, two-fold serial dilutions were made from H9+ cells from 500 cells/well to less than one cell/well (defined as a two-fold dilution beyond one cell/well). The results are shown in Table 8.

TABLE 8
Serial Dilution of H9+ Cells to Test Sensitivity of p24 Antigen Assay.

| Positive Control | | H9+ Cells | |
|---|---|---|---|
| Concentration ng/ml | Absorbance | Concentration | Absorbance |
| 0.25 | 1.03 | >8 cells/well | over |
| 0.125 | 0.55 | 8 cells/well | 1.53 |
| 0.0625 | 0.30 | 4 cells/well | 0.89 |
| 0.0313 | 0.15 | 2 cells/well | 0.53 |
| 0.0157 | 0.04 | <1 cell/well | 0.24 |
| 0.0 ng/ml | 0.03 | <1 cell/well | 0.10 |

Absorbance of known concentrations of p24 antigen in a commercial ELISA (Dupont) were compared with absorbance of cell lysates from an HIV-1 infected continuous cell line - H9.

These results indicate that the assay is extremely sensitive; it is able to detect p24 in<one cell/well down to 0.0157 ng/ml concentration.

Viral Purge from HIV+ Donor

The H9 studies indicated that positive selection of CD4+ cells combined with serial dilution could isolate a viral-free subpopulation of cells. The process can be monitored with great sensitivity by a commercial p24 assay. This process, however, does not address the purging of latent virus from the cells. In order for latent virus to proliferate, the host cell must be activated. The immobilized anti-CD3 system has proven to be an effective activator of these cells. After activation, the viral-free cells must be protected or they will soon become infected just as the indicator cells do in the p24 assay. Anti-CD4 mAb was used to protect uninfected CD4+ cells.

Material and Methods

Lymphocytes were Isolated from the AIDS patient following leukaphoresis as described above. A sample of unfractionated cells were tested for p24 in a co-cultivation test for 20 days. Similar samples were tested after macrophage adherence, CD4 positive selection and CDB positive selection. CD4+ cells were activated in 24-well plates on immobilized CD3 mAb. Soluble anti-CD28 was added to the medium and the cells were harvested after seven days. The CD4+ cells were then again labelled with anti-CD4 and positively selected for with GAM-coated immunomagnetic beads. The positively selected cells were relabelled with anti-CD3 and placed on GAM-coated 96-well plates at 25,000 cells/well. Anti-CD28 was added to the growth medium.

After seven days, supernatant from each well was tested for p24 antigen. All the negative wells were pooled and again subjected to CD4 positive selection with immunomagnetic beads. The positively selected cells were relabelled with anti-CD3 mAb and plated again at 25,000 cells per well. Anti-CD28 was added to the medium and the wells were tested for p24 again after seven days. Negative wells were again pooled and expanded as described previously for normal lymphocytes with the exception of only anti-CD28 and the addition of anti-CD4 (leu 3a, Becton Dickinson) to protect the cells from any residual virus. The cells were expanded to over ten million and a one-million cell aliquot was harvested for co-cultivation with indicator cells, p24 readings of cell lysate was taken after 20 days. Results are shown in Table 9.

TABLE 9
Viral-Purge of Lymphocytes from HIV+ Donor.
p24 Levels

| | |
|---|---|
| PBL (before adherence) | 0.32 ng |
| PBL (after adherence) | 0.28 ng |
| CD4+ | 0.24 ng |
| CD8+ | 0.00 ng |

Amount of p24 antigen recovered from a one million cell lysate of HIV+ cells before removal of macrophages by adherence to plastic T-flasks, after the removal of macrophages, after positive selection of CD4+ cells and CD8+ cells.

The CD4+ cells were plated at 25,000 cells per well of a 96-well plate and expanded for seven days on immobilized anti-CD3 mAb and soluble anti-CD28 mAb. Each well was then assayed for p24 antigen. Results are shown in Table 10.

TABLE 10
Detection of HIV-1 In Wells of Expanded
CD4+ Cells Purified from HIV+ Donor.

| | | # Greater than | |
|---|---|---|---|
| | # of Wells | Background | % Negative |
| Group 1 | 133 | 24 | 82% |
| Group 2 | 108 | 18 | 83% |
| Group 3 | 141 | 29 | 79% |

Amount of p24 antigen recovered from wells of 96-well plates with 25,000 CD4+ cells purified from the peripheral blood of an AIDS patient and expanded for seven days on immobilized anti-CD3 mAb and soluble anti-CD28 mAb. Each group represents the results of a separate purification from the same patient.

The percent negative wells was very consistent. The cells from the negative wells were pooled and propagated with immobilized anti-CD3 and anti-CD28, anti-CD4 was added to protect uninfected cells. All cells were plated at 2.5×10⁵ cells/well in 24-well plates. The number of CD4+ cells recovered after six days in culture is shown in Table 11.

TABLE 11
Pooled CD4+ Cells Purged of
Active and Latent Virus Expanded 6 Days.

| Day | Group 1 | Group 2 | Group 3 |
|---|---|---|---|
| 0 | 3.3 × 10⁶ | 2.1 × 10⁶ | 3.6 × 10⁶ |
| 6 | 12.4 × 10⁶ | 11.8 × 10⁶ | 11.4 × 10⁶ |

CD4+ cells purged of active and latent virus were expanded in 24-well plates. Cells were harvested and counted after six days in culture with immobilized anti-CD3 mAb and anti-CD28 mAb.

The cells from the 24-well plates were pooled and incubated in spinner flasks for three days. They were then relabelled with anti-CD4 and rosetted with GAM-coated immunomagnetic beads. 1×10⁶ positively selected cells were co-cultured with indicator cells for 20 days. The cell lysates for all three groups were negative for p24 (data not shown). These results demonstrate that this method is capable of producing a viral-free fraction of CD4+ cells from the peripheral blood of AIDS patients.

The cells from the three groups were pooled and relabelled with anti-CD3 mAb and inoculated into 2 GAM-coated cartridges of a min-hollow fiber device with 200 ng/ml of anti-CD28 mAb. After 21 days of culture, 1.7×10⁸ cells were harvested. Three days after harvest, the cells were relabelled with anti-CD3 mAb and inoculated into a single GAM-coated cartridge on the large scale device with 200 ng/ml of anti-CD28 mAb. After 21 days of culture, 1.1×10¹⁰ cells were harvested. Three days after harvest, these cells were relabelled with anti-CD3 mAb and inoculated into 8 GAM-coated cartridges on the large-scale device with 200 ng/ml of anti-CD28 mAb. After 18 days of culture, 6.4×10¹⁰ CD4+ cells were recovered. The cells were negative for p24.

CD4+ Functional Studies

To demonstrate that CD4+ cells isolated and propagated by this process were still capable of normal function, their ability to enhance NK activity was assessed. Patients with AIDS are known to have reduced NK function. Some reports have shown that exogenous IL-2 can significantly enhance NK-function of AIDS patients in-vitro. This study demonstrated that adding the expanded viral-purged CD4+ cells was effective.

Materials and Methods

The NK-sensitive cell line K562 was used as the target cell. The cells were chromium labelled by suspension at a concentration of 1×10⁷ cells/ml in cRPMI containing 100 μCi/ml of [⁵¹Cr] sodium chromate (New England Nuclear, Boston, Mass.) for 60 minutes at 37° C. The cells were then washed twice, resuspended at 5×10⁴ cells/ml in 100 μl aliquots into wells of round-bottomed 96-well plates.

Monocyte depleted lymphocytes from AIDS patients suspended at 5×10⁶ cells/ml were added to wells containing the target cells in 50 μl aliquots. An additional 50 μl of medium or CD4+ cells was added to each well such that the effector:target ratio without CD4+ cells was 50:1.

After a one hour incubation at 37° C. In five percent CO₂ at 100 percent humidity, the plates were centrifuged at 800×g for 12 minutes and 100 μl aliquots of each well were harvested and counted on a liquid scintillation counter. Percent lysis of each target cell was determined by the equation:

% lysis=cpmₜₑₛₜ−cpm_{control}/cpmₘₐₓ−cpm_{control}×100, where

cpmₜₑₛₜ indicates chromium counts per minute released in the presence of lymphocytes, cpm_{control} indicates release of the presence of medium alone, and cpmₘₐₓ indicates release in the presence of BRIS-35 detergent (Sigma, St. Louis, Mo.).

Each test was performed in quadruplicate. Significance of percent lysis was determined by comparing mean cpmₜₑₛₜ with mean cpm_{control} by student's t-test. Results are shown in Table 12.

TABLE 12
NK-Activity of Lymphocytes from AIDS Patient Supplemented
with Autologous, Viral-Purged CD4+ Cells.

| Results | % Lysis |
|---|---|
| AIDS lymphocytes alone | 26.2 ± 6.5% |
| AIDS lymphocytes + 1 IL-2 (10 U/ml) | 54.5 ± 6.8% |
| AIDS lymphocytes + CD4+ (1000) | 33.4 ± 7.0% |
| AIDS lymphocytes + CD4+ (5000) | 48.8 ± 3.5% |
| AIDS lymphocytes + CD4+ (10,000) | 64.6 ± 5% |
| AIDS lymphocytes + CD4+ (50,000) | 64.2 ± 9.5% |
| Normal lymphocytes alone | 60.2 ± 6.4% |
| Normal lymphocytes + IL-2 (10 U/ml) | 73.5 ± 6.5% |

NK-activity of a single AIDS patient after reconstruction with autologous, viral-purged CD4+ cells. The number of added cells is noted in parentheses. Results are expressed as the mean ± SE of quadruplicate samples.

The NK-activity of AIDS patients of 26.2±6.5% was significantly lower than the 60.2±6.4% for normal controls. The addition of IL-2 significantly increased NK-activity in normal and AIDS patients, but had a much greater effect in AIDS. The addition of 1,000 autologous CD4+ cells did not significantly increase NK-activity. Addition of 5,000 and 10,000 CD4+ cells significantly increased activity to normal levels. Addition of 50,000 CD4+ had the same effect as 10,000 cells.

These results evidence that the CD4+ cells isolated and expanded by this protocol are able to produce IL-2. These results also support the evidence that large numbers of these CD4+ cells infused back to the patient should restore immunological function.

Purification of HIV-Specific T-cells

HIV-specific class I-restricted T-cells are known to be present in the blood of AIDS patients; they are presumed to be a subset of CD8+, CD28+, CD 11⁻, CD25+ lymphocytes. These are in vivo activated (CD25+ same as IL2R+) Tc (CD28+ same as 9.3). To isolate these cells, a series of positive selection steps were conducted using CD8 (leu 2a, Becton Dickinson), CD28 (KOLT-2 gift from K. Sagawa), and CD25 (IL-2R, Coulter) mAbs and GAM-coated immunomagnetic beads.

Positive selection occurred in the following order: CD8, CD28, and finally, CD25. A subset of the isolated cells should be HIV-specific. The other in vivo T-cells in this group may also be of therapeutic importance; they may be specific for other adventitious agents afflicting the patient.

AIDS patients usually had a high percentage of CD25+ cells. In six patients tested, the mean CD25+ cells were 14±8% compared to six normal controls at 3±2.5%.

CD8+ Functional Studies

The CD8+ CD28+ CD25+ T-cells isolated from an AIDS patient and expanded to 5.3×10¹⁰ cells were tested for their ability to lyse HIV-infected autologous CD4+ lymphocytes. The target lymphocytes were expanded viral-free CD4+ cells from the same patient from whom the effector cells were isolated. The CD4+ cells were activated on immobilized anti-CD3 at 5×10⁵ cells/ml in one ml cRPMI on a 24-well plate. One ml of H9+ supernatant containing 10⁹ U/ml IL-2 was added to each well. The CD4+ cells were harvested from the wells after incubation at 37° C. in five percent CO₂ at 100 percent humidity for four days.

The cells were labelled with ⁵¹Cr using the same procedure as described for K562 target cells. All cells were plated in round-bottomed 96-well plates at effector:target ratios of 100:1, 50:1, and 25:1. Percent lysis was determined as described earlier. Each test was performed In triplicate. Results are shown in Table 13.

TABLE 13
CD8+, CD28+, CD25+ Killer T-Cells Isolated from HIV+
Patient, Ability to Lyse Autologous HIV Infected Cells

| Cell: Target Ratio | % Lysis |
|---|---|
| 100:1 | 21.0 ± 8.0% |
| 50:1 | 9.0 ± 3.5% |
| 25:1 | 3.5 ± 2.0% |

CD8+, CD28+, CD25+ Tc isolated from an AIDS patient were tested for their ability to lyse autologous CD4+ cells infected with HI-1. Percent lysis was calculated from a ⁵¹Cr-release assay.

These results indicate significant effector function. The low percentage lysis was probably due to a combination of a low percentage of targets infected with HIV (74 percent remained CD4+) and a high background.

Although the present invention has been described with reference to preferred embodiments, workers skilled in the art will recognize that changes may be made in form and detail without departing from the spirit and scope of the invention. Since modifications will be apparent to those of skill in this art, it is intended that this invention be limited only by the scope of the appended claims.

What is claimed is:

1. A method for selectively stimulating proliferation and differentiation of T lymphoid cells to generate a high density of clinically relevant numbers of T lymphoid cells, comprising:
collecting material comprising body fluid or tissue containing mononuclear cells from a mammal;
treating the cells are under conditions whereby ex vivo differentiation of the cells into Th2-like or Th2 cells is induced; and
contacting, in the absence of exogenous interleukin-2, the material with two or more activating proteins specific for cell surface proteins present on cells in the material and in an amount sufficient to induce ex vivo cell expansion, whereby the cells expand to at least about 10¹⁰ cells comprising predominantly Th2 or Th2-like cells.

2. The method of claim 1, further comprising purification of the expanded cells.

3. The method of claim 1, wherein the expanded cells are specific for a defined antigen.

4. The method of claim 1, wherein the expanded cells are predominantly Th2 cells.

5. The method of claim 1, wherein the cells are activated ex vivo in the presence of IL-4 with or without the presence of anti-gamma interferon and anti-IL-12 monoclonal antibodies to cause differentiation into Th2 cells.

6. The method of claim 1, wherein the immune cells are activated ex vivo in the presence of interferon-γ, whereby differentiation of Th2 cells are effected.

7. The method of claim 1, wherein the proteins specific for cell surface proteins are one or more monoclonal antibodies specific for immune cell surface proteins.

8. The method of claim 7, wherein the monoclonal antibodies are specific for CD3 or CD2, combined with any combination of monoclonal antibodies specific for one or more of the following: CD4, CD8, CD11a, CD27, CD28, CD44 and CD45RO.

9. The method of claim 1, wherein cell expansion is effected in a hollow fiber bioreactor.

10. The method of claim 1, wherein the cells are expanded to an excess of 10¹⁰ cells.

11. The method of claim 4, wherein the expanded cells are purified.

12. The method of claim 1, wherein the mammal is a human.

13. The method of claim 2, wherein the mammal is a human.

14. The method of claim 7, wherein the mammal is a human.

15. The method of claim 1, wherein the expanded cells are predominantly Th2 cells, whereby the resulting population has a Th2 or Th2-like cytokine profile.

16. The method of claim 2, wherein the expanded cells are predominantly Th2 cells, whereby the resulting population has a Th2 or Th2-like cytokine profile.

17. The method of claim 7, wherein the expanded cells are predominantly Th2 cells, whereby the resulting population has a Th2 or Th2-like cytokine profile.

18. A method for generating clinically relevant cell numbers of Th2 or Th2-like T lymphoid cells, comprising:
(a) collecting material containing mononuclear T lymphoid cells from a mammal;
(b) activating the T lymphoid cells to alter their cytokine production profile by causing differentiation of the cells into Th2 or Th2-like cells; and
(c) inducing cell proliferation and expanding the cells under conditions that produce at least about 10¹⁰ cells/liter of a homogeneous population of Th2 or Th2-like T lymphoid cells.

19. The method of claim 18, wherein the T lymphoid cells with altered cytokine profile are purified.

20. The method of claim 18, wherein the T lymphoid cells with altered cytokine profile are specific for a defined antigen.

21. The method of claim 18, wherein the T lymphoid cells are activated to differentiate into Th2 cells.

22. The method of claim 18, wherein the resulting population of expanded cells includes Th2-like cells.

23. The method of claim 22, wherein the cells are activated in the presence of IL-4 anti-gamma interferon antibodies and/or anti-IL-12 antibodies, whereby cells differentiate into Th2 cells

24. The method of claim 18, wherein the cells are expanded in the presence of two or more monoclonal antibodies.

25. The method of claim 24, wherein the monoclonal antibodies are specific for CD3 or CD2, combined with any combination of monoclonal antibodies specific for one or more of the following: CD4, CD8, CD11a, CD27, CD28, CD44 and CD45RO.

26. The method of claim 18, wherein the cells are expanded in a hollow fiber bioreactor.

27. The method of claim 18, wherein the cells are expanded to an excess of 10⁹ cells.

28. The method of claim 18, wherein the cells are expanded to an excess of 10¹⁰ cells.

29. A method for generating clinically relevant numbers of regulatory T lymphoid cells for autologous cell therapy, comprising:
(a) collecting material comprising body fluid or tissue containing mononuclear cells from a mammal;
(b) treating the cells to induce differentiation of mononuclear cells into Th2 or Th2-like cells; and
(c) contacting the resulting differentiated cells with two or more activating proteins specific for cell surface proteins present on the cells in an amount sufficient to induce ex vivo cell expansion, whereby clinically relevant numbers of regulatory cells for autologous cell therapy are generated.

30. The method of claim 29, wherein cells are purified from the material.

31. The method of claim 29, wherein the treating and contacting steps occur in the absence of exogenous cytokines or the contacting step occurs in the absence of exogenous cytokines.

32. The method of claim 29, wherein the cells are specific for a selected antigen.

33. The method of claim 29, wherein the resulting cells comprise CD4+ T-cells.

34. The method of claim 29, wherein the resulting cells are predominantly Th2 cells.

35. The method of claim 29, wherein the resulting cells comprise CD8+ T-cells.

36. The method of claim 29, wherein at step (b) the cells are treated with IL-4 with or without anti-gamma interferon antibodies and/or anti-IL-12 antibodies to cause differentiation into Th2 cells.

37. The method of claim 29, wherein the proteins specific for cell surface proteins are one or more monoclonal antibodies specific for immune cell surface proteins.

38. The method of claim 37, wherein the monoclonal antibodies are specific for CD3 or CD2, combined with any combination of monoclonal antibodies specific for one or more antigens selected from the group consisting of CD4, CD8, CD11a, CD27, CD28, CD44 and CD45RO.

39. The method of claim 29, wherein cell expansion is effected in a hollow fiber bioreactor.

40. The method of claim 29, wherein the cells are expanded to about 10⁹ cells or greater.

41. The method of claim 29, wherein the cells are expanded to about 10¹⁰ cells or greater.

42. The method of claim 29, wherein the expanded cells are predominantly Th2 cells.

43. The method of claims 29, wherein the expanded cells are contained in a volume of one liter or less.

44. The method of claim 29, wherein the expanded cells are contained in a volume of about 500 mls or less.

45. The method of claim 29, wherein the expanded cells are contained in a volume of about 250 mls or less.

46. The method of claim 29, wherein the expanded cells are predominantly Th2-like cell, wherein:
Th2-like cells are cells that produce a majority of Th2 cytokines.

47. A method for generating clinically relevant numbers of regulatory Th2, or Th2-like lymphoid cells for autologous cell therapy, comprising:
(a) collecting material comprising body fluid or tissue containing T lymphoid cells from a mammal;
(b) treating the cells to induce differentiation of some of the mononuclear cells into Th2 or Th2-like cells, wherein Th2-like cells are cells that produce a majority of Th2 cytokines; and
(c) contacting the cells with two or more activating proteins specific for cell surface proteins present on the cells in an amount sufficient to induce ex vivo cell expansion, whereby clinically relevant numbers of Th2 or Th2-like lymphoid cells are generated.

48. The method of claim 47, wherein cells are either purified or purged from the material.

49. The method of claim 47, wherein the treating or contacting steps occur in the absence of exogenous cytokines.

50. The method of claim 47, wherein the regulatory cells are specific for a defined antigen.

51. The method of claim 47, wherein the regulatory cells are CD4+ T-cells.

52. The method of claim 47, wherein the regulatory cells are CD8+ T-cells.

53. The method of claim 47, wherein the cells are treated with IL-4 with or without the presence of anti-gamma interferon monoclonal antibodies and/or anti-IL-12 monoclonal antibodies to cause the differentiation into Th2 cells.

54. The method of claim 47, wherein the proteins specific for cell surface proteins are one or more monoclonal antibodies specific for immune cell surface proteins.

55. The method of claim 54, wherein the monoclonal antibodies are specific for CD3 or CD2, combined with any combination of monoclonal antibodies specific for one or more of the following: CD4, CD8, CD11a, CD27, CD28, CD44 and CD45RO.

56. The method of claim 47, wherein cell expansion is effected in a hollow fiber bioreactor.

57. The method of claim 47, wherein the cells are expanded to an excess of 10⁹ cells.

58. The method of claim 47, wherein the cells are expanded to an excess of 10¹⁰ cells.

59. The method of claim 47, wherein the expanded cells are administered to a patient.

60. The method of claims 47, wherein the expanded cells are contained in a volume of about one liter or less.

61. The method of claim 47, wherein the expanded cells are contained in a volume of about 500 mls or less.

62. The method of claim 47, wherein the expanded cells are contained in a volume about 250 mls or less.

63. The method of claim 47, wherein the expanded cells are predominantly Th2 cells.

64. The method of claim 47, wherein the expanded cells are predominantly Th2-like cells.

65. The method of claim 47, wherein the expanded cells are predominantly Th2 cells.

66. The method of claim 1, wherein the 10¹⁰ cells that are predominantly Th2 cells are produced.

67. The method of claim 66, wherein the expanded cells are administered to a patient.

68. The method of claim 1, wherein the cells are at a density of 1×10⁸ cells/ml.

69. The method of claim 1, wherein density of the cells is at least 10⁹ cells/liter.

70. The method of claim 1, wherein density of the cells is at least 10¹⁰ cells/liter.

71. A composition, comprising predominantly Th2 or Th2-like cells produced by the method of claim 1.

72. The composition of claim 71, wherein the cells are at a density of 1×10⁸ cells/ml.

73. A method of treatment of diseases in which a Th1 cytokine profile predominates, comprising administering the composition of claim 71, thereby altering the ratio of Th1/Th2 cell.

74. The method of claim 72, wherein the disease is a chronic inflammatory disease, chronic infectious diseases or an autoimmune disease.

75. The method of claim 74, wherein the disease is selected from the group consisting of multiple sclerosis, rheumatoid arthritis, Crohn's Disease, autoimmune thyroid disease and inflammatory bowel disease

76. The method of claim 74, wherein the disease is selected from the group consisting of infections with human immunodeficiency virus, herpes simplex virus, cytomegalovirus or hepatovirus.

77. A composition produced by the method of claim 20.

78. A method of specific immunosuppression in organ and tissue transplant procedures or to provide immunoprotection in vaccination, comprising administering the composition of claim 20.

79. The method of claim 74, wherein the disease is rheumatoid arthritis, wherein the composition is produced by a method comprising:
collecting mononuclear cells from a rheumatoid arthritis patient;
expanding the cells under conditions whereby a composition containing an amount of Th2 cells sufficient to suppress or reduce the chronic inflammatory lesions of the arthritis; and
infusing the resulting composition of cells into the patient.

80. The method of claim 79, wherein the number Th2 cells is at least 10⁹.

81. The method of claim 79, wherein the cells are contained in a volume of 1 liter or less.

82. The method of claim 74, wherein the disease is multiple sclerosis, and the composition is produced by a method, comprising:
collecting mononuclear cells from a multiple sclerosis patient;
expanding the cells under conditions whereby a composition containing an amount of Th2 cells sufficient to ameliorate the symptoms or retard or stop the progression of multiple sclerosis; and
infusing the resulting composition of cells into the patient.

83. The method of claim 82, wherein the number of cells is at least 10⁹ cells.

84. The method of claim 82, wherein the cells are contained in a volume of 1 liter or less.

85. The method of claim 82, wherein the cells have a memory phenotype.

86. The method of claim 82, wherein the cells are specific for myelin or encephalitogenic epitopes of myelin antigens.

87. The method of claim 74, wherein the disease inflammatory bowel disease (IBD), and the composition is produced by a method, comprising:
collecting mononuclear cells from an IBD patient;
expanding the cells under conditions whereby a composition containing an amount of Th2 cells sufficient to ameliorate the symptoms or retard or stop the progression of the IBD; and
infusing the resulting composition of cells into the patient.

88. The method of claim 87, wherein the number of cells is at least 10⁹ cells.

89. The method of claim 87, wherein the cells are contained in a volume of 1 liter or less.

90. The method of claim 87, wherein the disease is Crohn's disease (CD) or ulcerative colitis (UC).

91. The method of claim 87, wherein the Th2 cells are express integrin, α4, β7.

92. A method for suppression transplant rejection, comprising:
collecting mononuclear cells from a patient prior to undergoing organ or tissue transplantation;
expanding the cells under conditions whereby a composition containing an amount of Th2 cells sufficient to prevent rejection of the transplanted organ or tissue; and
infusing the resulting composition of cells into the patient.

93. The method of claim 92, wherein the number of cells is at least 10⁹ cells.

94. The method of claim 92, wherein the cells are contained in a volume of 1 liter or less.

95. The method of claim 92, wherein the transplanted tissue are transplanted islets of Langerhans.

96. The method of claim 92, wherein the cells are specific for the alloantigens or for an antigen unique to the transplanted tissue or organ.

97. A method for treating insulin-dependent diabetes mellitus (IDDM), comprising:
collecting mononuclear cells from a patient diagnosed with IDDM or at high risk for developing IDDM;
expanding the cells under conditions whereby a composition containing an amount of Th2 cells sufficient to prevent or retard islet destruction; and
infusing the resulting composition of cells into the patient.

98. The method of claim 97, wherein the number of cells is at least 10⁹ cells.

99. The method of claim 97, wherein the cells are contained in a volume of 1 liter or less.

100. Cells produced by the method of claim 1 that have a CD⁺⁴ phenotype.

101. Cells produced by the method of claim 1 that have a CD⁺⁸ phenotype
